SEQUENCE LISTING (28 sequences)

SEQ ID NO: 1 - protein, 61 aa, Drosophila melanogaster
LRRRGRQTYTRYQTLELEKEFHTNHYLTRRRRIEMAHALCLTERQIKIWFQNRRMKLKKEI

SEQ ID NO: 2 - protein, 8 aa, Drosophila melanogaster
MNSYFEQA

SEQ ID NO: 3 - protein, 19 aa, Drosophila melanogaster
VRPSACTPDSRVGGYLDTS

SEQ ID NO: 4 - protein, 8 aa, Drosophila melanogaster
FYPWMAIA

SEQ ID NO: 5 - protein, 380 aa, Drosophila melanogaster
MNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 6 - protein, 4 aa, Artificial sequence
  Synthetic construct
GGGS

SEQ ID NO: 7 - protein, 5 aa, Artificial sequence
  Synthetic construct
GGGGS

SEQ ID NO: 8 - protein, 4 aa, Artificial sequence
  Synthetic construct
GGSG

SEQ ID NO: 9 - protein, 5 aa, Artificial sequence
  Synthetic construct
GGSGG

SEQ ID NO: 10 - protein, 5 aa, Artificial sequence
  Synthetic construct
GSGSG

SEQ ID NO: 11 - protein, 5 aa, Artificial sequence
  Synthetic construct
GSGGG

SEQ ID NO: 12 - protein, 5 aa, Artificial sequence
  Synthetic construct
GGGSG

SEQ ID NO: 13 - protein, 5 aa, Artificial sequence
  Synthetic construct
GSSSG

SEQ ID NO: 14 - protein, 7 aa, Artificial sequence
  Synthetic construct
TSGSGSH

SEQ ID NO: 15 - protein, 1233 aa, Artificial sequence
  Synthetic construct
MKNRNRMIVNCVTASLMYYWSLPALAEQSSSEIKIVRDEYGMPHIYANDTWHLFYGYGYVVAQDRLFQMEMARRSTQGTVAEVLGKDFVKFDKDIRRNYWPDAIRAQIAALSPEDMSILQGYADGMNAWIDKVNTNPETLLPKQFNTFGFTPKRWEPFDVAMIFVGTMANRFSDSTSEIDNLALLTALKDKYGVSQGMAVFNQLKWLVNPSAPTTIAVQESNYPLKFNQQNSQTAALLPRYDLPAPMLDRPAKGADGALLALTAGKNRETIAAQFAQGGANGLAGYPTTSNMWVIGKSKAQDAKAIMVNGPQFGWYAPAYTYGIGLHGAGYDVTGNTPFAYPGLVFGHNGVISWGSTAGFGDDVDIFAERLSAEKPGYYLHNGKWVKMLSREETITVKNGQAETFTVWRTVHGNILQTDQTTQTAYAKSRAWDGKEVASLLAWTHQMKAKNWQEWTQQAAKQALTINWYYADVNGNIGYVHTGAYPDRQSGHDPRLPVPGTGKWDWKGLLPFEMNPKVYNPQSGYIANWNNSPQKDYPASDLFAFLWGGADRVTEIDRLLEQKPRLTADQAWDVIRQTSRQDLNLRLFLPTLQAATSGLTQSDPRRQLVETLTRWDGINLLNDDGKTWQQPGSAILNVWLTSMLKRTVVAAVPMPFDKWYSASGYETTQDGPTGSLNISVGAKILYEAVQGDKSPIPQAVDLFAGKPQQEVVLAALEDTWETLSKRYGNNVSNWKTPAMALTFRANNFFGVPQAAAEETRHQAEYQNRGTENDMIVFSPTTSDRPVLAWDVVAPGQSGFIAPDGTVDKHYEDQLKMYENFGRKSLWLTKQDVEAHKESQEVLHVQRTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 16 - protein, 718 aa, Artificial sequence
  Synthetic construct
MASTPKANVPKIDVSPLFGDNMEEKMKVARAIDAASRDTGFFYAVNHGVDVKRLSNKTREFHFSITDEEKWDLAIRAYNKEHQDQIRAGYYLSIPEKKAVESFCYLNPNFKPDHPLIQSKTPTHEVNVWPDEKKHPGFREFAEQYYWDVFGLSSALLRGYALALGKEEDFFSRHFKKEDALSSVVLIRYPYLNPIPPAAIKTAEDGTKLSFEWHEDVSLITVLYQSDVANLQVEMPQGYLDIEADDNAYLVNCGSYMAHITNNYYPAPIHRVKWVNEERQSLPFFVNLGFNDTVQPWDPSKEDGKTDQRPISYGDYLQNGLVSLINKNGQTTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 17 - protein, 785 aa, Artificial sequence
  Synthetic construct
MAVADWEEARGRMLLDPTVVNLNTGSGGPLPRSAFERVTGFRAHLAAEPMDFLLREVPALLWQARESLARLIGGDPLRLALATNVTAAVNLVASSLRLEAPGEILLSDDEYTPMRWCWERVARRHGLELRTFRLPELPSDPAEITAAAVAAMGPRTRLFFFSHVVSTTGLILPAAELCEEARARGITTVVDGAHAPGFLDLDLSRIPCDFYAGSGHKWLLAPTGVGFLHLAPGRLEELEPTQVSWAYEPPEGSGPPAARDRFGSTPGLRRLECEGTRDICPWLATPESIDFQAELGPGAIRARRRELTDHARRLLADRPGRTLLTPDSPELSGGMVAYRLPPGTDAAELRRGLWERFRIEAAVAEQPPGPVLRISANFYTTEEEIDRLADALDALTGETSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 18 - protein, 698 aa, Artificial sequence
  Synthetic construct
MDTTVPTFSLAELQQGLHQDEFRRCLRDKGLFYLTDCGLTDTELKSAKDLVIDFFEHGSEAEKRAVTSPVPTMRRGFTGLESESTAQITNTGSYSDYSMCYSMGTADNLFPSGDFERIWTQYFDRQYTASRAVAREVLRATGTEPDGGVEAFLDCEPLLRFRYFPQVPEHRSAEEQPLRMAPHYDLSMVTLIQQTPCANGFVSLQAEVGGAFTDLPYRPDAVLVFCGAIATLVTGGQVKAPRHHVAAPRRDQIAGSSRTSSVFFLRPNADFTFSVPLARECGFDVSLDGETATFQDWIGGNYVNIRRTSKATSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 19 - protein, 705 aa, Artificial sequence
  Synthetic construct
MADTPVPIFNLAALREGADQEKFRECVTGMGVFYLTGYGAGDKDHRLATDTAMDFFANGTEAEKAAVTTDVPTMRRGYSALEAESTAQVTRTGSYTDYSMSFSMGISGNVFPSPEFERVWTEYFDKLYAAAQETARLVLTASGGYDAEIVGSLDELLDADPVLRLRYFPEVPEHRSAEHEPRRMAPHYDLSIITFIHQTPCANGFVSLQAEIGGELVSLPVVEDAVVVMCGAMAPLATQGALPAPRHHVRSPGAGMREGSDRTSSVFFLRPTTDFSFSVAKARSYGLAVDLDMETATFGDWIGTNYVTMHAKNEPQAGTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 20 - protein, 831 aa, Artificial sequence
  Synthetic construct
MLPSAQVARLKPDPFPPSLSPIPHGAVTFAALAPCHNLPIFSSRQMLRDSLTYSHTSPTMSPQIANRFEASLDAQDIARISLFTLESGVILRDVPVAYKSWGRMNVSRDNCVIVCHTLTSSAHVTSWWPTLFGQGRAFDTSRYFIICLNYLGSPFGSAGPCSPDPDAEGQRPYGAKFPRTTIRDDVRIHRQVLDRLGVRQIAAVVGASMGGMHTLEWAFFGPEYVRKIVPIATSCRQSGWCAAWFETQRQCIYDDPKYLDGEYDVDDQPVRGLETARKIANLTYKSKPAMDERFHMAPGVQAGRNISSQDAKKEINGTDSGNSHRAGQPIEAVSSYLRYQAQKFAASFDANCYIAMTLKFDTHDISRGRAGSIPEALAMITQPALIICARSDGLYSFDEHVEMGRSIPNSRLCVVDTNEGHDFFVMEADKVNDAVRGFLDQSLMTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 21 - protein, 762 aa, Artificial sequence
  Synthetic construct
MATAPLVIRAEGGPRERGARYGAAARDKVRMSLGSYERVYAKFAGMSWTEATTLAGEFVPVLSGWRPEYLEEIRGIAEGAGVSFEDVLALNLRTEIMFSGKARRLAEETGIRPPAECTAFCDLRGPEPLAGQNWDWIPFAHDTVVVLETAPERGPRWVSVVEAGLLAKFGMNSAGIAVVTNALVTSLDVGAPGVPYHMLLRALLESESMAAAEEVLRGADRASSANYLLVGEGRGVDAEARPGGVGDLGWRRTGAEAVLVHCNHFVCAEAEFSSSGTDHGPEIMPDSLLRRERALSLAGRMPEPSVDGWHEVLTDHENFPAAVCCHPHPERTALEQGSTVASVVMEPERRLMHLVLGRPCEAPRVTYDYGTFLAGTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 22 - protein, 877 aa, Artificial sequence
  Synthetic construct
MQPAMMMFSSKYWARRGFSLDSAVPEEHQLLGSSTLNKPNSGKNDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGITSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 23 - protein, 867 aa, Artificial sequence
  Synthetic construct
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERETSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 24 - protein, 594 aa, Artificial sequence
  Synthetic construct
MSTQSTHPLKPEAPRLPPGIPESPSCQRRHTLPASEFRCLTPEDAVSAFEIEREAFISVLGVCPLYLDEIRHFLTLCPELSLGWFEEGCLVAFIIGSLWDKERLMQESLTLHRSGGHIAHLHVLAVHRAFRQQGRGPILLWRYLHHLGSQPAVRRAALMCEDALVPFYERFSFHAVGPCAITVGSLTFMELHCSLRGHPFLRRNSGCTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 25 - protein, 732 aa, Artificial sequence
  Synthetic construct
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARKTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 26 - protein, 826 aa, Artificial sequence
  Synthetic construct
MSKYFEKVSKIKYEGPKSNNPYAFKFYNPEEVIDGKTMEEHLRFSIAYWHTFTADGTDQFGKATMQRPWNHLTDPMDIAKARVEAAFEFFDKINAPFFCFHDRDIAPEGDTLRETNKNLDIIVAMIKDYLKTSKTKVLWGTANLFSNPRFVHGASTSCNADVFAYSAAQVKKALEITKELGGQNYVFWGGREGYETLLNTDMELELDNFARFLHMAVDYAKEIGFEGQFLIEPKPKEPTKHQYDFDVANVLAFLRKYDLDKYFKVNIEANHATLAAHDFQHELRYARINGVLGSIDANTGDMLLGWDTDQFPTDIRMTTLAMYEVIKMGGFDKGGLNFDAKVRRASFEPEDLFLGHIAGMDAFAKGFKVAYKLVKDGVFDKFIEERYASYKDGIGADIVSGKADFKSLEKYALEHSEIVNKSGRQEMLESILNQYLFTETSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 27 - protein, 1038 aa, Artificial sequence
  Synthetic construct
MSESTPEVSSSYPPPAHFAEHANARAELYREAEEDRLAFWAKQANRLSWTTPFTEVLDWSGAPFAKWFVGGELNVAYNCVDRHVEAGHGDRVAIHWEGEPVGDRRTLTYSDLLAEVSKAANALTDLGLVAGDRVAIYLPLIPEAVIAMLACARLGIMHSVVFGGFTAAALQARIVDAQAKLLITADGQFRRGKPSPLKAAADEALAAIPDCSVEHVLVVRRTGIEMAWSEGRDLWWHHVVGSASPAHTPEPFDSEHPLFLLYTSGTTGKPKGIMHTSGGYLTQCCYTMRTIFDVKPDSDVFWCTADIGWVTGHTYGVYGPLCNGVTEVLYEGTPDTPDRHRHFQIIEKYGVTIYYTAPTLIRMFMKWGREIPDSHDLSSLRLLGSVGEPINPEAWRWYRDVIGGGRTPLVDTWWQTETGSAMISPLPGIAAAKPGSAMTPLPGISAKIVDDHGDPLPPHTEGAQHVTGYLVLDQPWPSMLRGIWGDPARYWHSYWSKFSDKGYYFAGDGARIDPDGAIWVLGRIDDVMNVSGHRISTAEVESALVAHSGVAEAAVVGVTDETTTQAICAFVVLRANYAPHDRTAEELRTEVARVISPIARPRDVHVVPELPKTRSGKIMRRLLRDVAENRELGDTSTLLDPTVFDAIRAAKTSGSGSHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

SEQ ID NO: 28 - protein, 932 aa, Artificial sequence
  Synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDLAYWDEDEHFFIVGRLKSLIKYKGYQVAPAELESILLQHPNIFDAGVAGLPDDDAGELPAAVVVLEHGKTMTEKEIVDYVASQVTTAKKLRGGVVFVDEVPKGLTGKRDARKIREILIKAKKGGKSKLGHMNSYFEQASGFYGHPHQATGMAMGSGGHHDQTASAAAAAYRGFPLSLGMSPYANHHLQRTTQDSPYDASITAACNKIYGDGAGAYKQDCLNIKADAVNGYKDIWNTGGSNGGGGGGGGGGGGGAGGTGGAGNANGGNAANANGQNNPAGGMPVRPSACTPDSRVGGYLDTSGGSPVSHRGGSAGGNVSVSGGNGNAGGVQSGVGVAGAGTAWNANCTISGAAAQTAAASSLHQASNHTFYPWMAIAGKIRSDLTQYGGISTDMGKRYSESLAGSLLPDWLGTNGLRRRGRQTYTRYQTLELEKEFHTNHYLTRRRRIEMAHALCLTERQIKIWFQNRRMKLKKEIQAIKELNEQEKQAQAQKAAAAAAAAAAVQGGHLDQ

(19) United States
(12) Patent Application Publication
(10) Pub. No.: US 2024/0116992 A1
(43) Pub. Date: Apr. 11, 2024

(54) PROTEIN-BASED MATERIAL FOR BIOCATALYSIS

(71) Applicant: Bondwell Technologies LP, College Station, TX (US)

(72) Inventors: David W. Howell, College Station, TX (US); Sarah E. Bondos, College Station, TX (US); Rebecca M. Booth, College Station, TX (US); Christi Parham, College Station, TX (US); Dat P. Truong, College Station, TX (US); Rebecca E. Skouby, College Station, TX (US)

(73) Assignee: Bondwell Technologies LP, College Station, TX (US)

(21) Appl. No.: 18/263,833

(22) PCT Filed: Feb. 2, 2022
(86) PCT No.: PCT/US2022/014944

§ 371 Date: Aug. 1, 2023

Related U.S. Application Data
(60) Provisional application No. 63/144,755, filed on Feb. 2, 2021.

(51) Int. Cl. C07K 14/435; C12N 9/02; C12N 9/10; C12N 9/84; C12N 9/88; C12N 9/90; C12N 9/92; C12N 11/02; C12P 17/18; C12P 37/00

(52) U.S. Cl. CPC C07K 14/43581; C12N 9/0004; C12N 9/0071; C12N 9/1007; C12N 9/1029; C12N 9/84; C12N 9/88; C12N 9/90; C12N 9/92; C12N 11/02; C12P 17/182; C12P 37/00; C12Y 114/16004; C12Y 114/20001; C12Y 121/03001; C12Y 201/01004; C12Y 203/01164; C12Y 203/01175; C12Y 203/01194; C12Y 305/01011; C12Y 401/01028; C12Y 501/01017; C12Y 503/01018; C07K 2319/735

(57) ABSTRACT

Provided herein are compositions and methods for stabilizing enzymes. Also provided are systems, compositions and methods for using the stabilized enzymes to produce a desired compound.

### PRIOR RELATED APPLICATION

This application claims the benefit of U.S. Provisional Application No. 63/144,755 filed on Feb. 2, 2021, which is hereby incorporated by reference in its entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 1298582_seglist.txt, created on Feb. 2, 2022, having a size of 105 KB, and is filed concurrently with the specification. The sequence listing contained in this ASCII formatted document is part of the specification and is herein incorporated by reference in its entirety.

### BACKGROUND

The majority of drugs and industrial chemicals are produced by traditional chemical syntheses that require significant resources and facilities. Synthesis reactions involve multiple chemical reactions, each with its own isolation and purification train. Each isolation/purification step typically requires different solvents which account for up to 80-90% of the total mass in a process, resulting in substantial waste. Significant reliance on corrosive and/or toxic reagents further complicates waste disposal. Traditional chemical synthesis employs inefficient chemical reactions, assisted by extremes of pH, temperature, and pressure that require large equipment and energy inputs. Finally, drug molecules typically contain a number of often densely clustered, polar functional groups such as bases, weak protic acids, amides, amines, alcohols, hydrogen-bond donors or acceptors, and nitrogen-containing heterocycles. Maintaining reaction fidelity often requires additional protection and deprotection steps, lengthening the process and adding solvents, catalysts, waste and significant expense. Flow chemistry, the most cutting-edge chemical synthesis technology, uses continuous flow inside a sealed reactor. Multiple reactions and purification steps are combined (telescoped) to create complex molecules in one continuous stream, reducing solvent use and waste generation. Even with flow chemistry, the combination of resources required and toxic waste produced makes chemical synthesis expensive.

To overcome these limitations, the multi-billion dollar biocatalysis industry uses enzymes to produce fine chemicals, pharmaceuticals, and other industrially relevant chemicals. Unlike their chemical catalyst counterparts, enzymes are active in aqueous environments at ambient temperature and pressure, eliminating the need for large volumes of dangerous solvents, extensive facilities to create and contain hazardous conditions, and mechanisms to dispose of toxic waste. However, despite this great potential, industrial applications have been hampered, as enzymes are easily degraded and can rapidly lose activity during storage. Despite efforts to improve enzyme stability, for example, by immobilizing enzyme(s), it is still difficult to predict stabilization conditions for each enzyme, thus rendering these methods inadequate. For example, covalent chemical cross-linking is the most widely used technique for enzyme immobilization. However, covalent immobilization has several limitations, including, i) covalent cross-linking typically requires harsh conditions such as high temperatures, high voltages, exposure to organic chemicals, and/or extreme pHs, which can compromise enzyme structure and activity, ii) enzyme activity is lost due to inefficient crosslinking of the enzyme to the support leading to a significant failure rate (˜20%), and iii) because every enzyme has different surface chemistry, the protocol has to be individually optimized for every enzyme. Therefore, compositions and methods for immobilizing and/or stabilizing a wide variety of enzymes are necessary.

### SUMMARY

Provided herein are systems, compositions and methods for safely and reliably immobilizing and stabilizing a wide array of enzymes. The methods described herein significantly reduce costs, while eliminating time-consuming optimization processes. The compositions and methods provided herein not only stabilize enzymes during use, but also allow dry storage of the enzymes at room temperature. Furthermore, the systems, compositions and methods described herein facilitate multiple, linked enzymatic reactions for production of pharmaceuticals, chemicals, and other valuable compounds via biocatalysis.

Provided herein is a system for catalytically producing a compound, wherein the system includes one or more solid matrices including a fusion protein, wherein the fusion protein includes at least one Ubx protein and an enzyme or catalytic fragment thereof. In some embodiments, the system includes, two, three, four, five, six, seven, or more solid matrices.

In some embodiments, the system includes one or more reaction chambers including the one or more solid matrices. In some embodiments, the two, three, four, five, six, seven, or more reaction chambers are in fluid communication with each other. In some embodiments, the enzyme or catalytic fragment thereof in each of the two, three, four, five, six, seven, or more solid matrices is an enzyme in a biocatalytic cascade.

In some embodiments, the enzyme or catalytic fragment thereof, of each fusion protein, is an oxidoreductase, a transferase, a hydrolase, a lyase, a ligase, an isomerase, or a catalytic fragment thereof. In some embodiments, the enzyme or catalytic fragment thereof, of each fusion protein in the system, is the same or different. In some embodiments, the fusion protein in each chamber is immobilized. In some embodiments, the enzyme of catalytic fragment thereof of the fusion protein in each chamber is stabilized.

Also provided is a method for producing a desired compound including: (a) contacting a first reaction chamber of one or more reaction chambers of any of the systems described herein with a substrate, to catalytically produce one or more reaction products; and (b) (i) recovering the desired compound from the one or more reaction products produced in the first reaction chamber; or (ii) continuously or non-continuously flowing the one or more reactions products from the first reaction chamber to one or more subsequent reaction chambers for further catalysis; and recovering the desired compound from the one or more subsequent reaction chambers. In some embodiments, one or more cofactors are added to the first reaction chamber and/or to the one or more subsequent reaction chambers. In some methods, the desired compound is a pharmaceutical, a fine chemical, or a bulk chemical.

Also provided is a fusion protein including at least one Ubx protein and an enzyme or catalytic fragment thereof wherein the enzyme or catalytic fragment thereof is an oxidoreductase, a transferase, a hydrolase, a lyase, a ligase, an isomerase or a catalytic fragment thereof.

Also provided is a solid matrix comprising any of the fusion proteins described herein. Further provided is a reaction chamber including any of the fusion proteins described herein.

### BRIEF DESCRIPTION OF THE DRAWINGS

The present application includes the following figures. The figures are intended to illustrate certain aspects and/or features of the methods described herein, and to supplement any description(s) of the compositions and methods. The figures do not limit the scope of the methods, unless the written description expressly indicates that such is the case.

FIG. 1 is an exemplary schematic of a fusion protein containing an enzyme fused to an Ultrabithorax (Ubx) protein. As shown in FIG. 1, the fusion protein can optionally include a peptide tag and linker.

FIG. 2 shows an exemplary cascade system pathway. The synthesis of β-lactam antibiotics involves two common reactions to produce Isopenicillin N from amino acids. Cephalosporin C is produced in two subsequent steps, and Penicillin G in one step. Amoxicillin is derived from Penicillin G in one step. The enzymes catalyzing each reaction are labeled adjacent to the reaction arrow. The cofactors for each reaction are provided in Table 2.

FIGS. 3A and 3B illustrate successful genetic fusion of β-Lactam enzymes with Ubx. (A) Western blot of Isopenicillin N acyltranferase-Ultrabithorax (IPNAT-Ubx, 80 kDa), Isopenicillin N synthase-Ultrabithorax (IPNS-Ubx, 78 kDa), Isopenicillin N Epimerase-Ultrabithorax (IPNE-Ubx, 83 kDa), Deacetoxycephalosporin C synthetase-Ultrabithorax (DAOCS-Ubx, 75 kDa), Deacetylcephalosporin C synthetase-Ultrabithorax (DACS-Ubx, 75 kDa), Deacetylcephalosporin C acetyltransferase-Ultrabithorax (DACOAT-Ubx, 89 kDa). (B) Western blot of Penicillin G acylase-Ultrabithorax (PGA-Ubx 136 kDa).

FIG. 4 is an exemplary melatonin synthesis pathway that produces melatonin from tryptophan. The enzymes catalyzing each reaction are labeled above each reaction arrow. The cofactors for each reaction are provided in Table 3.

FIGS. 5A-5D illustrate successful genetic fusion of melatonin pathway enzymes with Ubx. The Western blot detected N-AcetylTransferase-Ultrabithorax (SNAT-Ubx, 63 kDa) (FIG. 5A), Acetylserotonin O-methyltransferase-Ultrabithorax (ASOMT-Ubx, 78 kDa) (FIG. 5B), Tryptophan 5 Hydroxylase-Ultrabithorax (T5H-Ubx, 106 kDa) (FIG. 5C), and 5-HydroxyTryptophan decarboxylase-Ultrabithorax (HTDC-Ubx, 104 kDa) (FIG. 5D).

FIGS. 6A and 6B show that Penicillin G Acylase, fused to Ubx, catalyzed the reaction from Penicillin G to Amoxicillin in two steps. Both steps demonstrated kinetics consistent with the literature.

FIG. 7 illustrates glucose/xylose conversion to fructose/xylulose via glucose isomerase.

FIG. 8 is a Western blot showing successful fusion of glucose isomerase to Ubx (GI-Ubx, 89 kDa).

FIG. 9 shows that glucose concentration increases over time when GI-Ubx materials are exposed to fructose.

FIG. 10 shows that Penicillin G Acylase-Ubx material retained its enzymatic activity as compared to fresh material, when subjected to conditions mimicking storage and/or shipping conditions at temperatures fluctuating between 10° C.-30° C.

FIG. 11 shows that Isopenicillin N Synthase, fused with Ubx, stored dry and at room temperature for two days, retained activity. This demonstrates reusability of enzyme-UBX fused material.

FIG. 12 illustrates the reusability of enzyme-Ubx materials. HTDC-Ubx materials were used to carry out the reaction of 5-HydroxyTryptophan to Serotonin and then stored dry or in PBS at room temperature for 48 hours. The reaction was run again using fresh substrate and the activity compared to the first run. Material stored in PBS retained 84% activity, while material stored dry only retained 61% activity. Therefore, storage in an aqueous buffer, like PBS, can contribute to material stability. Interestingly, there is no statistical difference between dry storage for 48 hours vs dry storage for 2 weeks, indicating that degradation likely occurs primarily during use and not during storage.

FIG. 13 shows that the methods of immobilization described throughout allow enzymes fused with Ubx to be stored at ambient temperature and retain activity (83%), even after seven months (FIG. 15). In fact, accelerated aging experiments showed that activity was retained in Luciferase-Ubx materials after 3 years (28% of fresh). Three years was simulated using an accelerated aging technique of placing the luciferase-Ubx material in an environmental chamber set to 40° C. and 55% humidity for 11 weeks. For comparison, soluble luciferase was used as a control and showed no signal after aging.

FIG. 14 is a schematic of a 3-step cascade reaction. Each enzyme(s)-Ubx membrane is sandwiched inside a reaction chamber.

FIG. 15 shows single pot synthesis of N-acetylserotonin using Tryptophan 5 Hydroxylase-Ubx, 5-HydroxyTryptophan decarboxylase-Ubx, and Serotonin Acetylase (SNAT)-Ubx materials in a single reaction cascade.

FIGS. 16A-16C show analysis of melatonin synthesis by liquid chromatography-mass spectrometry. A) Control reaction showing no product without N-Acetylserotonin O-methyltransferase (ASMOT)-Ubx materials. B) Detection of peak at 12.82 when ASMOT-Ubx materials are added to the reaction. C) Structure determination.

### DETAILED DESCRIPTION

The following description recites various examples of the present methods. No particular example is intended to define the scope of the methods. Rather, these are non-limiting, exemplary methods. The description is to be read from the perspective of one of ordinary skill in the art; therefore, information well known to the skilled artisan is not necessarily included.

Unless defined otherwise, all technical and scientific terms used herein have the same meaning as commonly understood by one of ordinary skill in the art to which this disclosure belongs. All patents, patent applications, and publications referred to throughout the disclosure herein are incorporated by reference in their entirety.

As used in the specification and the appended claims, the singular forms “a,” “an,” and “the” include plural referents unless the context clearly dictates otherwise. For example, reference to “a polypeptide” or “the polypeptide” may include a plurality of polypeptides.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Ranges can be expressed herein as from one particular value, and/or to another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

As used throughout, the term “nucleic acid sequence,” “nucleotide sequence,” or “polynucleotide sequence” refers to deoxyribonucleic acids (DNA) or ribonucleic acids (RNA) and polymers thereof in either single- or double-stranded form. It is understood that when an RNA is described, its corresponding DNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides described herein also encompass all forms of sequences including, but not limited to, single-stranded forms, double-stranded forms, hairpins, stem-and-loop structures, and the like.

Unless otherwise indicated, a particular nucleic acid sequence also implicitly encompasses modified variants thereof, alleles, orthologs, SNPs, and complementary sequences as well as the sequence explicitly indicated.

The terms “polypeptide,” “peptide,” and “protein” are used interchangeably herein to refer to a polymer of amino acid residues. The terms encompass amino acid chains of any length, including full-length proteins, wherein the amino acid residues are linked by covalent peptide bonds. The definition also includes fragments and variants of any polypeptide described herein. The protein may be produced by host cells, for example, in bacterial, yeast, insect, or mammalian cells using methods known to those of skill in the art.

The terms “solid phase,” “support,” “scaffold,” and “matrices” are used herein interchangeably, and refer to the solid material (e.g., Ubx material structure formed by two or more, i.e., a plurality of Ubx polypeptides) that provides a physical structure to immobilize an enzyme or a catalytic fragment thereof. Any of the matrices described herein can be formed by two, three, four, five, six, seven, eight, nine, ten, twenty, thirty, forty, fifty, sixty, seventy, eighty, ninety, or one hundred or more Ubx polypeptides.

The term “fusion protein” as used herein refers to a protein containing two or more polypeptides that are derived from different proteins but produced as a single polypeptide from a polynucleotide including a nucleotide sequence encoding both proteins and, in some embodiments, a linking sequence under control of a single promoter. The two or more polypeptides in the fusion proteins described herein, e.g., a Ubx protein or fragment thereof, and an enzyme or catalytic fragment thereof, are conjugated or linked, optionally, via a linker.

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains. In some embodiments, the linker can be at least two, three, four, five, six, seven, eight, nine, ten amino acids or greater, in length. Exemplary flexible linkers include but are not limited to GGGS (SEQ ID NO:6), GGGGS (SEQ ID NO: 7), GGSG (SEQ ID NO 8), GGSGG (SEQ ID NO: 9), GSGSG (SEQ ID NO: 10), GSGGG (SEQ ID NO: 11), GGGSG (SEQ ID NO: 12), GSSSG (SEQ ID NO: 13), and TSGSGSH (SEQ ID NO: 14).

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

The term “enzyme” or “biocatalyst” refers to a biological catalyst that accelerates the rate of a chemical reaction. A “catalytic fragment” refers to a region or portion of an enzyme that interacts with its substrate to cause the enzymatic reaction. As used herein, the enzyme or biocatalyst used in any of the compositions and methods provided herein can be a full-length enzyme or a catalytic fragment thereof. A catalytic fragment can be a polypeptide fragment derived from the full-length sequence of an enzyme, wherein the fragment has at least 50%, 60%, 70%, 60%, 90%, 95%, 99% of the catalytic activity of the enzyme from which the fragment is derived. Examples of enzymes include, but are not limited to, oxidoreductases, transferases, hydrolases, lyases, isomerases, ligases, and translocases.

Oxidoreductases typically catalyze oxidation-reduction reactions where electrons are transferred. These electrons are usually in the form of hydride ions or hydrogen atoms. When a substrate is being oxidized it is the hydrogen donor. The most common name used is a dehydrogenase and sometimes reductase will be used. An oxidase is referred to when the oxygen atom is the acceptor. Examples of oxidoreductases include, but are not limited to dehydrogenases, oxidases, oxygenases, peroxidases, and reductases.

Transferases catalyze group transfer reactions. The transfer occurs from one molecule that will be the donor to another molecule that will be the acceptor. Typically, the donor is a cofactor that is charged with the group about to be transferred. Examples of transferases include, but are not limited to, C₁-transferases, glycosyltransferases, aminotransferases, and phosphotransferases.

Hydrolases catalyze reactions that involve hydrolysis. This usually involves the transfer of functional groups to water. When the hydrolase acts on amide, glycosyl, peptide, ester, or other bonds, they not only catalyze the hydrolytic removal of a group from the substrate but also a transfer of the group to an acceptor compound. Examples of hydrolases include, but are not limited to esterases, glycosidases, lipases, proteases, peptidases, and amidases.

Lyases catalyze reactions where functional groups are added to break double bonds in molecules or the reverse where double bonds are formed by the removal of functional groups. Examples of lyases include, but are not limited to aldolases, decarboxylases, and dehydratases.

Isomerases catalyze reactions that transfer functional groups within a molecule so that isomeric forms are produced. These enzymes allow for structural or geometric changes within a compound. Sometimes the interconversion is carried out by an intramolecular oxidoreduction. In this case, one molecule is both the hydrogen acceptor and donor, so there's no oxidized product. An exemplary isomerase is phosphoglucose isomerase, which moves a chemical group inside the same substrate, for converting glucose 6-phosphate to fructose 6-phosphate. Examples of isomerases include, but are not limited to, epimerases, racemases, and intramolecular transferases.

Ligases are used in catalysis where two substrates are ligated and the formation of carbon-carbon, carbon-sulfide, carbon-nitrogen, and carbon-oxygen bonds due to condensation reactions. These reactions are coupled to the cleavage of ATP. Exemplary ligases include, but are not limited to, C—C, C—N, C—O, and C—S ligases.

Exemplary enzymes that can be used in any of the compositions, methods and systems described herein are provided in Table 1. Table 1 provides the name of the enzyme, the International Union of Biochemistry and Molecular Biology (IUBMB) classification of the enzyme, the class of enzyme and an exemplary non-limiting application for each enzyme. It is understood that the full-length or a fragment of each of the enzymes listed in Table 1 can be used in a single biocatalytic reaction or as reaction in a series (i.e., two or more) continuous or non-continuous biocatalytic reactions. The enzymes described throughout can be derived from any species, for example, from a bacterial species, a fungal species, a mammalian species, or a plant species, to name a few. One of skill in the art would understand that each enzyme class described herein is characterized by a catalytic domain. Therefore, one of skill in the art could use routine sequence alignment tools to identify other enzymes in each class described herein.

TABLE 1

| | IUBMB | | |
|---|---|---|---|
| Enzyme | number | Class | Application |
| Aminopeptidase | 3.4.11 | Hydrolase | Food |
| Amylase | 3.2.1.1 | Hydrolase | Food |
| Asparaginase | 3.5.2.1 | Hydrolase | Food |
| Catalase | 1.11.1.6 | Oxidoreductase | Food |
| Cellulase | 3.2.1.4 | Hydrolase | Food/biofuel |
| Glucose Oxidase | 1.1.3.4 | Oxidoreductase | Food |
| Lactase | 3.2.1.23 | Hydrolase | Food |
| Pectin lyase | 4.2.2.10 | Lyase | Food |
| Phosphodiesterase | 3.1.4.1 | Hydrolase | Food |
| nitrile hydratase | 4.2.1.84 | Lyase | fine/bulk |
| | | | chemical |
| nitrilase | 3.5.5.1 | Hydrolase | fine/bulk |
| | | | chemical |
| lactoaldehyde reductase | 1.1.1.77 | Oxidoreductase | fine/bulk |
| | | | chemical |
| lactoaldehyde | 1.2.1.22 | Oxidoreductase | fine/bulk |
| dehydrogenase | | | chemical |
| glycerol dehydratase | 4.2.1.30 | Lyase | fine/bulk |
| | | | chemical |
| oxidoreductase-isoenzyme | 1.1.1.202 | Oxidoreductase | fine/bulk |
| | | | chemical |
| epoxide hydrolases | 3.3.2.3 | Hydrolase | fine/bulk |
| | | | chemical |
| cyclodextrin | 2.4.1.19 | Transferase | fine/bulk |
| glycosyltransferase | | | chemical |
| cytochrome P450 | 1.14.13.8 | Oxidoreductase | fine/bulk |
| monooxygenases | | | chemical |
| glycolate oxidase | 1.1.3.1 | Oxidoreductase | |
| alcohol dehydrogenases | 1.1.5.5 | Oxidoreductase | biofuel |
| phenylalanine ammonia | 4.3.1.24 | Lyase | biofuel |
| lyase | | | |
| alkane hydroxylase | 1.14.15.3 | Oxidoreductase | biofuel |
| methane monooxygenase | 1.14.13.25 | Oxidoreductase | biofuel |
| laccases | 1.10.3.2 | Oxidoreductase | bioremediation |
| phenylalanine | 1.4.1.20 | Oxidoreductase | drug |
| dehydrogenase | | | |
| transaminase | 2.6.1.1 | Transferase | drug |
| alanine racemase | 5.1.1.1 | Isomerase | drug |
| DNA Ligase | 6.5.1.1 | Ligase | pharmaceutical |

In some embodiments, one or more enzymes are selected from the group consisting of ACV synthase, isopenicillin N synthase, isopenicillin N acyltransferase, penicillin G acylase, isopenicillin N epimerase, deacetoxycephalosporin C synthase, deacetoxycephalosporin C hydrolase, deacetylcephalosporin C acetyltransferase, tryptophan hydroxylase, 5-hydroxytryptophan decarboxylase, serotonin N acyltransferase, acylserotonin O-methyltransferase, glucose isomerase, and acetyl-coA synthase.

The term “Ubx material” or “Ubx materials” refers to the biomaterial made from the self-assembly of the Drosophila melanogaster transcription factor Ultrabithorax (Ubx) (See, for example, U.S. Patent Application Publication No. US2010/0143436), or any of the improved synthetic versions of Ultrabithorax described in US Patent Application Publication No. US2018/0222949A1. In some examples, the Ubx material contains two or more self-assembled Ubx protein molecules. In any of the Ubx materials described herein, the Ubx protein can include an amino acid sequence selected from the group consisting of the amino acid sequence set forth under GenBank Accession No. AAN13717, GenBank Accession No. AAN13718, GenBank Accession No. AAN13719, GenBank Accession No. AAF55355, GenBank Accession No. AAF55356, and GenBank Accession No. AAS65158, or a fragment thereof.

In some embodiments, a full-length Ubx protein (for example, SEQ ID NO: 5) is used to create a fusion protein. In other embodiments, the Ubx protein that is used to produce a Ubx fusion protein is a fragment of a full-length Ubx protein containing a Ubx homeodomain, for example, a Ubx protein fragment containing SEQ ID NO:1

(LRRRGRQTYTRYQTLELEKEFHTNHYLTRRRRIEMAHALCLTERQIKI
WFQNRRMKLKKEI).

For example, and not to be limiting, the Ubx protein or polypeptide can be a fragment of SEQ ID NO: 5 that comprises SEQ ID NO: 1. For example, the fragment can comprise amino acids 280-345 of SEQ ID NO: 5, amino acids 285-345 of SEQ ID NO: 5, amino acids 275-345 of SEQ ID NO:5, amino acids 270-345 of SEQ ID NO:5, amino acids 265-345 of SEQ ID NO:5, amino acids 260-345 of SEQ ID NO:5, amino acids 255-345 of SEQ ID NO:5 or amino acids 250-345 of SEQ ID NO: 5, amino acids 245-345 of SEQ ID NO: 5, amino acids 240-345 of SEQ ID NO: 5, amino acids 235-345 of SEQ ID NO: 5, amino acids 230-345 of SEQ ID NO: 5, amino acids 225-345 of SEQ ID NO: 5, amino acids 220-345 of SEQ ID NO: 5, amino acids 215-345 of SEQ ID NO: 5, amino acids 210-345 of SEQ ID NO: 5, amino acids 205-345 of SEQ ID NO: 5, amino acids 200-345 of SEQ ID NO: 5, amino acids 195-345 of SEQ ID NO: 5, amino acids 190-345 of SEQ ID NO: 5, amino acids 180-345 of SEQ ID NO: 5, amino acids 175-345 of SEQ ID NO: 5, amino acids 170-345 of SEQ ID NO: 5, amino acids 165-345 of SEQ ID NO: 5, amino acids 160-345 of SEQ ID NO: 5, amino acids 155-345 of SEQ ID NO: 5, amino acids 153-345 of SEQ ID NO: 5, or amino acids 150-345 of SEQ ID NO: 5.

In other embodiments, the Ubx fusion protein includes a Ubx protein fragment containing SEQ ID NO:1 and SEQ ID NO:2 (MNSYFEQA). In other embodiments, the Ubx fusion protein includes a Ubx protein fragment containing SEQ ID NO:3 (VRPSACTPDSRVGGYLDTS) and/or SEQ ID NO:4 (FYPWMAIA). In some embodiments, the Ubx fusion protein includes one or more of the Ubx protein fragments described herein, and is not the full-length Ubx protein. Nucleic acid sequences encoding any of the polypeptides described herein are also provided. Polypeptide and nucleotide sequences having at least 60% identity to any of the polypeptide sequences provided herein can also be used in the compositions and methods provided herein. In some embodiments, the polypeptide and nucleotide sequences have at least 60% identity to any of the polypeptide sequences provided herein and retain at least one activity of the polypeptide or nucleic acid from which it was derived, for example, catalytic activity or self-assembly of Ubx polypeptides.

The term “identity” or “substantial identity”, as used in the context of a polypeptide or polynucleotide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. Provided herein are amino acid sequences having at least 60%, 65%, 70%, 75%, 80%, 85%, 90%, 91%, 92%, 93%, 94%, 95%, 96%, 97%, 98%, or 99% to any sequence provided herein (for example, SEQ ID NOs. 1-28).

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, about 20 to 50, about 20 to 100, about 50 to about 200, or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

The term “functional material” or “enzyme-Ubx material” refers to a Ubx biomaterial made from a fusion protein containing an enzyme or catalytic fragment, and a Ubx protein or a fragment thereof. In some embodiments, the Ubx biomaterial comprises two or more self-assembled fusion proteins (for example, two, three, four, five, six, seven, eight, nine, ten, twenty, thirty, forty, fifty, sixty, seventy, eighty, ninety, or one hundred or more) containing an enzyme or a catalytic fragment thereof and a Ubx protein. In some embodiments, two or more self-assembled fusion proteins form one or more solid matrices of the systems described herein.

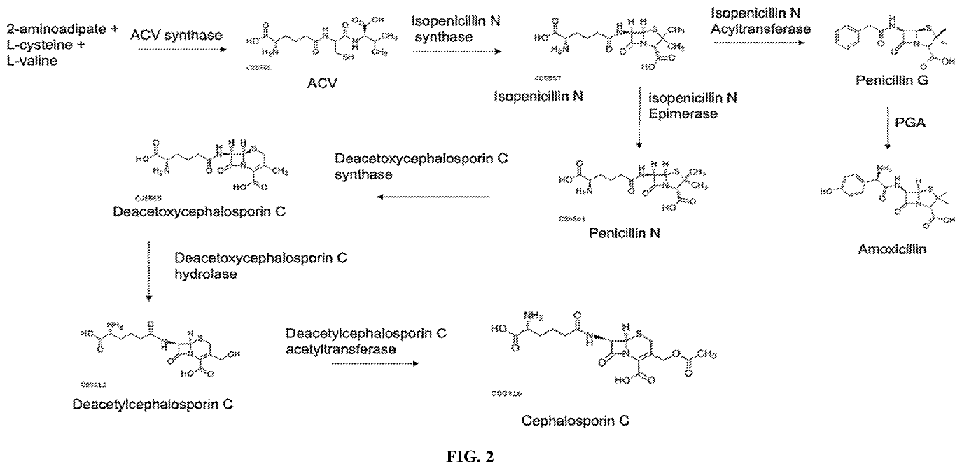

As used herein the term “biocatalytic cascade” refers to a multi-step, i.e., two or more catalytic or enzymatic steps, for producing a desired compound. For example, the biocatalytic cascade can include two, three, four, five, six, seven, eight, nine, ten, or more steps. In some embodiments, the reaction product(s) catalyzed by a step in the cascade flows into the next catalytic step of the cascade, either continuously or non-continuously. For example, the reaction product(s) of the first step of the biocatalytic cascade can flow into the second step of the biocatalytic cascade, and optionally, the reaction product of the second step can flow into a third step of the biocatalytic cascade. The methods described herein can be used to perform all of the steps of a biocatalytic cascade or subsets of steps. It is understood that a reaction product obtained via biocatalysis or other means can be used as substrate in any of the biocatalytic processes described herein. For example, using the methods described herein, amoxicillin can be produced using a four step process, as illustrated in FIG. 2, or amoxicillin can be produced by obtaining isopenicillin N via biocatalytic by other means and using a two step process (i.e., an isopenicillin acyltranferase reaction and a PGA reaction), as shown in FIG. 2.

As used herein, the term “purified” or “purify” refers to separating a substance from at least some of the components (e.g., impurities or contaminants) with which it was associated when initially produced. For example, compounds are purified by removal of contaminating enzymes, reagents etc. Purified substances can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

### DETAILED DESCRIPTION

### Systems

Provided herein are systems for producing a desired compound using either a continuous or non-continuous cascade of biocatalysts, i.e., enzymes. In some embodiments, the system includes one or more solid matrices comprising a fusion protein including at least one Ubx protein and an enzyme or catalytic fragment thereof. In some embodiments, the system includes two, three, four, five, six, seven or more solid matrices.

In some embodiments, the system includes one or more reaction chambers including the one or more solid matrices. In some embodiments, the two, three, four, five, six, seven, eight, nine, ten, eleven, twelve, or more reaction chambers are in fluid communication with each other.

For example, and not to be limiting, the system can include (i) a first reaction chamber containing a first solid matrix, wherein the solid matrix includes a fusion protein containing at least one Ubx protein and an enzyme or catalytic fragment thereof; and (ii) a second reaction chamber containing a second solid matrix, wherein the second reaction chamber is in fluid communication with the first reaction chamber, wherein the second solid matrix contains a second fusion protein containing at least one Ubx protein and an enzyme or catalytic fragment thereof, and wherein the enzyme or catalytic fragment thereof of the second fusion protein is the same or different from the enzyme or catalytic fragment thereof of the first fusion protein.

In some embodiments, the system further includes a third reaction chamber containing a third solid matrix, wherein the third reaction chamber is in fluid communication with the second reaction chamber, wherein the third solid matrix includes a third fusion protein containing at least one Ubx protein and an enzyme or catalytic fragment thereof, and wherein the enzyme or catalytic fragment thereof of the third fusion protein is the same or different from the enzyme or catalytic fragment thereof of the first fusion protein or the second fusion protein.

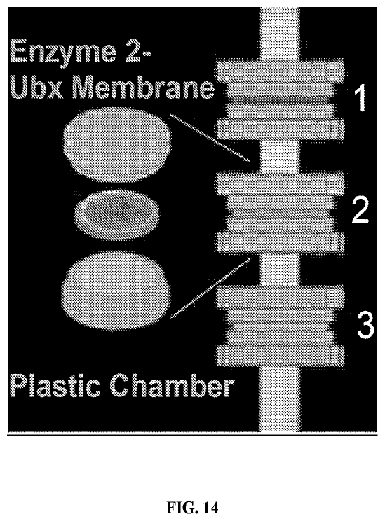

It is understood that the system can further comprise additional reaction chambers, for example, a third, fourth, fifth, sixth, seventh, eighth, ninth, tenth chamber etc., wherein each of the chambers comprises a solid matrix containing a fusion protein containing at least one Ubx protein and an enzyme or catalytic fragment thereof. Optionally, each reaction chamber in any of the systems described herein is in fluid communication with the preceding reaction chamber, for example, a second reaction chamber in fluid communication with a first reaction chamber, a third reaction chamber in fluid communication with a second reaction chamber, a fourth reaction chamber in fluid communication with a third reaction chamber etc. In some embodiments, each of the reaction chambers in the system are in fluid communication with a preceding reaction chamber. In some embodiments, the two or more reaction chambers (i.e., two, three, four, five, six, seven reaction chambers, etc.) are in a bioreactor. The reaction chambers described herein can be any vessel in which the reagents, liquid, gases, and/or temperature can be contained, under conditions that allow the reaction to occur. The reaction chamber can be a plastic chamber, a glass chamber, a quartz chamber, or a metal chamber, to name a few. In some embodiments, the reaction chamber can be a quartz cuvette placed in a temperature-controlled holder, or an acrylic syringe filter housing with plastic tubing connecting each chamber (FIG. 14).

As used herein “continuously” or “continuous” refers to a system or method wherein the reaction product(s) of a reaction (for example, a reaction in a reaction chamber) flows into a subsequent reaction as part of a continuous process or continuously flowing stream. Such a continuously flowing stream or process is not limited to two or more reaction chambers in fluid communication with each other, as any of the systems described herein can include dispensing means, pumps, mixers and downstream units.

As used herein, “non-continuously” or “non-continuous” refers to a system or method wherein the reaction product(s) of a first reaction (for example, a reaction in a reaction chamber) are flowed or transferred to a subsequent reaction chamber in the system, wherein the subsequent reaction chamber is not in fluid communication with the first reaction chamber.

In some embodiments, the enzyme or catalytic fragment thereof in each of the two, three, four, five, six, seven, or more solid matrices is an enzyme in a biocatalytic cascade.

In some embodiments, the enzyme or catalytic fragment thereof of each fusion protein is an oxidoreductase, a transferase, a hydrolase, a lyase, a ligase, an isomerase or a catalytic fragment thereof. In some embodiments, the enzyme or catalytic fragment thereof of each fusion protein in the system is the same or different. In some embodiments, the fusion protein in each reaction chamber is immobilized. In some embodiments, the enzyme or catalytic fragment thereof of the fusion protein in each chamber is stabilized.

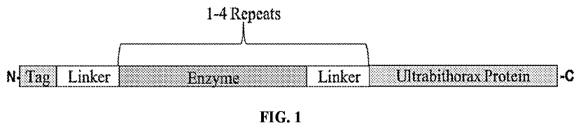

The systems described herein include a biomaterial for immobilization of an enzyme. In some embodiments, the biomaterial includes a Ubx protein or a fragment thereof. The biomaterials provided herein have a functional property that is imparted through fusion of an enzyme or catalytic fragment thereof to a Ubx protein or a fragment thereof. The enzyme or catalytic fragment can be of natural or synthetic origin. In some embodiments, the enzyme is selected from the group consisting of an oxidoreductase, a transferase, a hydrolase, a lyase, an isomerase and a ligase. In some embodiments, multimeric fusions containing two or more, for example, two-eighteen copies, of the same enzyme or catalytic fragment thereof (FIG. 1) can be used. In other embodiments, multimeric enzymes containing two or more, for example, two-eighteen different monomeric fragments can be used. Functional variants of enzymes or catalytic fragments can be used in any of the embodiments described herein.

Provided herein is a fusion protein containing (1) at least one Ubx protein or a fragment thereof, and (2) an enzyme or catalytic fragment thereof. In some embodiments, the enzyme or catalytic fragment thereof is an oxidoreductase, a transferase, a hydrolase, a lyase, a ligase, an isomerase, or a catalytic fragment thereof. Also provided is a solid matrix containing any of the fusion proteins described herein. Optionally, the solid matrix comprising the fusion proteins can be affixed to or in contact with a solid support such as, for example, a filter paper, a gel, beads, or a resin. Further provided are one or more reaction chambers, wherein each of the reaction chambers contains a fusion protein described herein. In some embodiments, the reaction chamber includes a solid matrix containing, or composed of, the fusion protein. In some embodiments, the fusion protein is immobilized and/or stabilized in the reaction chamber. In some embodiments, one or more reaction chambers contain one or more fusion proteins described herein, wherein the enzyme or catalytic fragment thereof in each chamber is a different enzyme or catalytic fragment thereof. In some embodiments, two or more different enzymes are part of a biocatalytic cascade. In some embodiments, any of the enzyme containing-Ubx materials or solid matrices containing a fusion protein described herein retain at least 50%, 60%, 70%, 80%, 90%, 95%, or 99% of its enzymatic activity for about one month, two months, three months, four months, five months, six months, seven months, eight months, nine months, ten months, eleven months, one year, two years or longer after immobilization of the enzyme or catalytic fragment thereof in the Ubx material or solid matrix, as compared to a control, for example, the corresponding non-Ubx containing enzyme or materials comprising same.

In some embodiments, any of the enzyme containing-Ubx materials or solid matrices containing a fusion protein described herein is thermostable at temperatures ranging from about 20° C. to about 50° C. for at least one day, two days, three days, four days, five days, six days, one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen weeks, nineteen weeks or twenty weeks. In some embodiments, any of the enzyme containing-Ubx materials or solid matrices containing a fusion protein described herein is thermostable at temperatures ranging from about 20° C. to about 50° C. for at least one month, two months, three months, four months, five months, six months, seven months, eight months, nine months, ten months, eleven months, one year, 1.5 years, two years, 2.5 years, three years, 3.5 years, or four years.

In some embodiments, any of the enzyme containing-Ubx materials or solid matrices containing a fusion protein described herein is thermostable at temperatures ranging from about 20° C. to about 50° C. for at least one day, two days, three days, four days, five days, six days, one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen weeks, nineteen weeks, or twenty weeks as compared to the thermostability of the non-Ubx containing enzyme or a matrix comprising a non-Ubx containing enzyme. In some embodiments, any of the enzyme containing-Ubx materials or solid matrices containing a fusion protein described herein is thermostable at temperatures ranging from about 20° C. to about 50° C. for at least one month, two months, three months, four months, five months, six months, seven months, eight months, nine months, ten months, eleven months, one year, 1.5 years, two years, 2.5 years, three years, 3.5 years, or four years, as compared to the thermostability of the non-Ubx containing enzyme or a matrix comprising a non-Ubx containing enzyme.

In some embodiments, any of the fusion proteins described herein or matrices comprising the fusion protein has increased stability as compared to the enzyme or catalytic fragment when it is not fused to the Ubx protein. This increase can be an increase of at least 10%, 20%, 30%, 40%, 50%, 60%, 70%, 80%, 90%, 100%, 200%, 300%, 400%, 500% or greater. In some embodiments, the increase is at least a 2-fold, 5-fold, 10-fold, 20-fold, 25-fold, 30-fold, 35-fold, 40-fold, 45-fold, 50-fold, 60-fold, 70-fold, 80-fold, 90-fold, or 100-fold increase in stability. In some embodiments, the increase in enzyme stability can be an increase in stability at increased temperature (e.g., thermostability), exposure to high (e.g., a pH of about 9 or greater) or low pH (e.g., a pH of about 5 or lower), or stability in the presence of organic solvents.

In any of the embodiments provided herein, the functional property of biocatalysis is imparted through a fusion protein including an enzyme or catalytic fragment thereof and a Ubx protein or a fragment thereof. These fusion proteins are made using art-recognized techniques for expressing non-naturally occurring or synthetic proteins. For example, the nucleic acid encoding the functional fusion protein (i.e., a nucleic acid encoding a fusion protein that includes an enzyme or a catalytic fragment thereof and a Ubx protein or a fragment thereof) is cloned into a plasmid for expressing the protein, generally 3′ to a DNA sequence encoding a peptide tag to facilitate protein purification. In some embodiments, a linker is placed at the 3′ end of the nucleic acid sequence encoding the enzyme or catalytic fragment. The length and sequence of this linker can vary. For example, the linker can be from about two to about twenty amino acids in length. In some embodiments, the linker contains alternating repeats of glycine and serine. When a linker is present, the nucleic acid encoding the Ubx protein or a fragment thereof is located at the 3′ end of the linker followed by one or more stop codons, the only one(s) in the gene fusion. Expression of the fused gene in live bacterial and/or yeast cells produces the fused protein, which can be used to make functionalized materials. See, for example, U.S. Patent Publication No. 2018/0222949 for methods of making fusion proteins and functionalized materials.

In any of the embodiments provided herein, large, complex proteins can be immobilized via protein fusion (i.e., fusion of the protein to Ubx), thus eliminating the need for chemical crosslinkers. In the protein fusion approach, a nucleic acid sequence encoding a desired enzyme is placed adjacent to a nucleic acid encoding Ubx or a fragment thereof. In bacteria, as an example, this fusion produces, for example, an Enzyme-Ubx protein fusion as a single polypeptide. After the enzyme and Ubx segments of the fusion protein independently fold, the Ubx portion of the fusion protein drives materials formation. Only the compositions described herein can successfully incorporate proteins up to 160 kDa. Most enzymes are significantly larger than 35 kDa, therefore the compositions and systems provided herein allow for reliable immobilization of enzymes while maintaining enzymatic stability throughout a biocatalytic process. In particular, the immobilization and stabilization of multiple enzymes that are part of a multi-step biocatalytic process was unexpected.

### Methods

Also provided is a method for producing a desired compound including: (a) contacting a first reaction chamber of the one or more reaction chambers of any of the systems described herein with a substrate, to catalytically produce one or more reaction products; and (b) (i) recovering the desired compound from the one or more reaction products produced in the first reaction chamber; or (ii) continuously or non-continuously flowing the one or more reactions products from the first reaction chamber to one or more subsequent reaction chambers for further catalysis; and recovering the desired compound from the one or more subsequent reaction chambers. In some embodiments, one or more cofactors are added to the first reaction chamber and/or to the one or more subsequent reaction chambers. In some methods, the desired compound is a pharmaceutical (for example, an antibiotic (e.g., β-lactam antibiotics), a statin, a hormone, etc.), a chemical (for example, a fine chemical or a bulk chemical (e.g., an acetyl-CoA derived chemical)), a drug, a food additive (e.g., fructose or glucose), or a biofuel (e.g. ethanol). It is understood that any compound that can be made using multi-step biocatalysis can be produced using any of the methods and systems provided herein.

In some embodiments, the contacting step is performed by dispensing a liquid or solution containing the substrate into the first reaction chamber. In some embodiments a dispensing means is in fluid communication with the first reaction chamber of the system.

In some embodiments, the method includes (a) contacting a buffered solution containing precursors (i.e., a substrate) and co-factors with a solid matrix containing any of the fusion proteins describe herein; (b) allowing the enzyme to interact with a substrate in the solution; (c) recovering the desired product(s) from the reaction chamber; and (d) continuously or non-continuously flowing the reaction product(s) to a subsequent chamber for (1) further biocatalytic conversion or (2) purification of the final desired product. In the methods described herein, the enzyme is immobilized onto a protein-based biomaterial, i.e., a Ubx material, by fusing the enzyme or catalytic fragment thereof to a Ubx protein or a fragment thereof. In the methods provided herein, the solid matrix is composed of Ubx materials as a solid support. In any of the embodiments described herein, the biomaterial is made from the self-assembly of Ubx proteins, fusion proteins containing a Ubx protein, or fragments thereof.

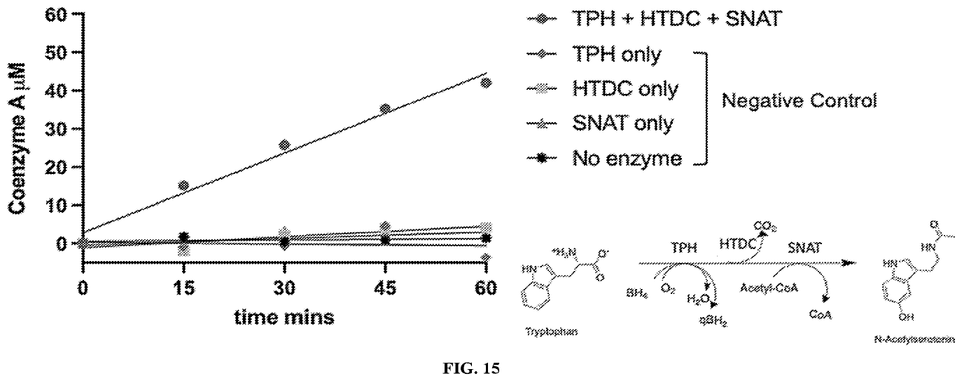

In some embodiments, biocatalytic cascades are used to synthesize pharmaceuticals, chemicals, and other valuable compounds by connecting the appropriate series of reaction chambers, and adding substrate, and optionally, any cofactors, to the first reaction chamber (FIG. 15). Reaction product(s) will flow from the first reaction chamber to one or more subsequent reaction chambers until the desired compound is obtained from the last reaction chamber. It is understood that the methods described herein can include one or more catalytic reactions of a biocatalytic cascade, but are not limited to including all of the reactions in a biocatalytic cascade, i.e., the entire catalytic cascade. In some embodiments, cofactors are also added to one or more subsequent chambers. Each disk or matrix can be rinsed, dried and re-used as needed to manufacture one or more compounds. Any of the methods described herein can further include one or more purification steps after obtaining the reaction product(s) from one or more reaction chambers, for example, the last reaction chamber, to produce the desired compound. In some embodiments, purification includes concentration of the desired compound. Concentration can be carried out by any suitable means of concentration known in the art, for example, using by one or more of liquid chromatography, centrifugation, microfiltration, ultrafiltration, or nanofiltration, to name a few.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### Drug Synthesis

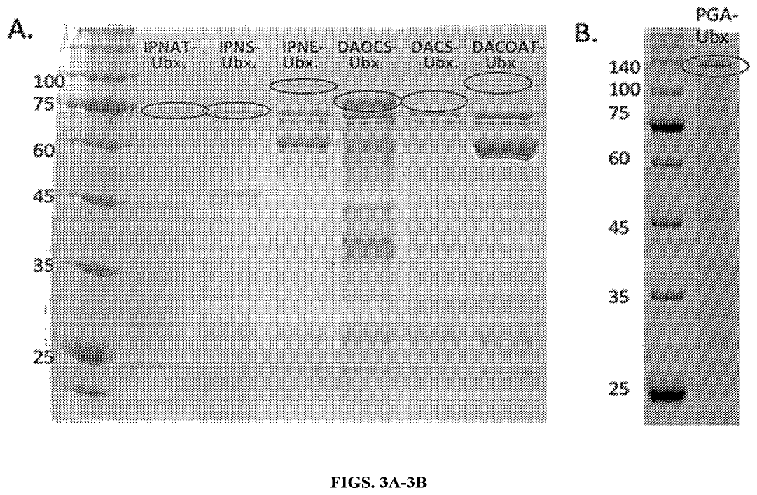

Two natural pathways were used to produce reaction products from base substrates. The first pathway immobilized seven (7) of eight (8) enzymes (Isopenicillin N acyltranferase (IPNAT, 80 kDa), Isopenicillin N synthase (IPNS, 78 kDa), Isopenicillin N Epimerase (IPNE, 83 kDa), Deacetoxycephalosporin C synthetase (DAOCS, 75 kDa), Deacetylcephalosporin C synthetase (DACS, 75 kDa), Deacetylcephalosporin C acetyltransferase (DACOAT, 89 kDa) and Penicillin G acylase (PGA 136 kDa)) to Ubx materials to produce Cephalosporin C, Penicillin G, and Amoxicillin, three members of the valuable P3-lactam class of antibiotics (FIGS. 2 and 3). The substrates and cofactors for this reaction series are all commercially available as follows: ascorbic acid from JT Baker (Radnor, PA); Coenzyme A from Sigma (St. Louise, MO); Tetrahydrobiopterin from Millipore (Burlington, MA); Ammonium Ferric Sulfate from Sigma; FeSO₄ and MgSO₄ from VWR (Radnor, PA); pyridoxal phosphate from Sigma; deacetylcephalosporin C, Isopenicilin N, and Penicillin N synthesized by Enamine (Monmouth Jct, NJ); deacetoxycephalosporin C synthesized using the methods described herein; and ACV, 2-aminoadipate, cysteine, valine, penicillin G, and serotonin pathway substrates purchased from Sigma. The cofactors and buffers used in this exemplary biocatalytic cascade are set forth in Table 2.

TABLE 2
Cofactors for production of Cephalosporin C, Penicillin G, and Amoxicillin

| Enzyme | Substrate | Co-Factor | Product | Buffer |
|---|---|---|---|---|
| ACV synthase | 5 mM 2- | O₂ | ACV | 5 mM ATP |
| | aminoadipate | Fe²⁺ | | 50 mM HEPES, pH 7.0 |
| | 5 mM L-cysteine | | | 30° C. |
| | 5 mM L-valine | | | 300 mM NaCl |
| | | | | 2 mM DTT |
| | | | | 5 mM MgCl₂ |
| Isopenicillin N | 2 mM ACV | O₂ | Isopenicillin N | 1.2 mM TCEP |
| synthase | | | | 25 μM ascorbate |
| | | | | 10 μM NH₄Fe(II)SO₄ |
| | | | | 50 mM Phosphate, 7.0 |
| | | | | 25° C. |
| Isopenicillin N | Isopenicillin N | 0.2 mM | Penicillin G | 100 mM Tris, pH |
| acyltransferase | | Phenylacetyl- | | 8.0 |
| | | CoA | | 25° C. |
| Penicillin G | Penicillin G | p- | Amoxicillin | dimethylaminobenz |
| Acylase | | hydroxyphenyl | | ylaldehyde |
| | | glycine methyl | | 50 mM phosphate, |
| | | ester | | pH 7.0 |
| | | | | 30° C. |
| Isopenicillin N | Isopenicillin N | | Penicillin N | 50 mM Phosphate, 8.0 |
| epimerase | | | | |
| Deacetoxycephalosporin | Penicillin N | 0.8 mM α- | Deacetoxycephalosporin | 50 mM Tris, pH 7.4 |
| C synthase | | ketoglutarate | C | 10 mM DTT |
| | | | | 80 mM (NH4)₂SO4 |
| | | | | 10 mM ascorbate |
| | | | | 30° C. |
| Deacetoxycephalosporin | Deacetoxycephalosporin | 0.8 mM α- | Deacetylcephalosporin | 50 mM Tris, pH 7.4 |
| C hydrolase | C | ketoglutarate | C | 10 mM DTT |
| | | | | 80 mM (NH₄)₂SO₄ |
| | | | | 0.04 mM FeSO₄ |
| | | | | 30° C. |
| Deacetylcephalosporin | Deacetylcephalosporin | 5 mM Acetyl- | Cephalosporin C | 100 mM Tris, pH 7.5 |
| C acetyltransferase | C | CoA | | 4 mM MgSO4 |
| | | | | 1 mM DNTB |
| | | | | 30° C. |

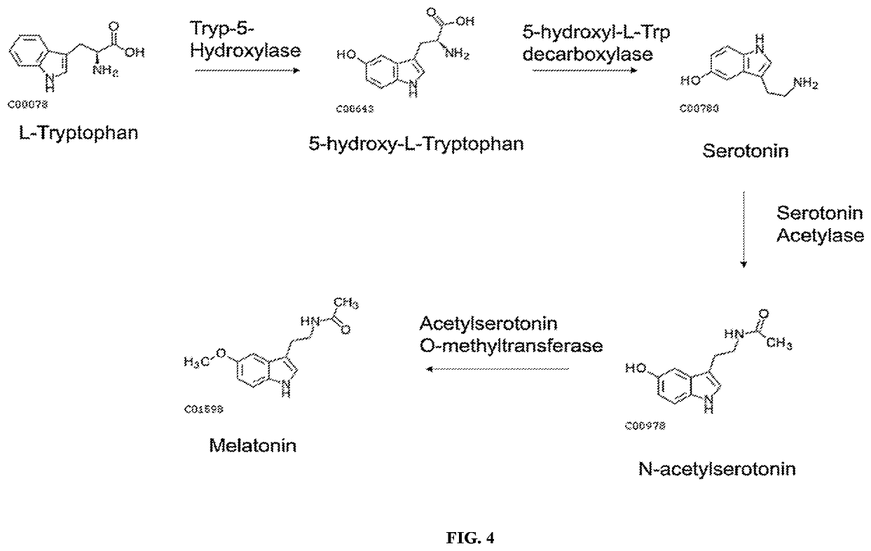

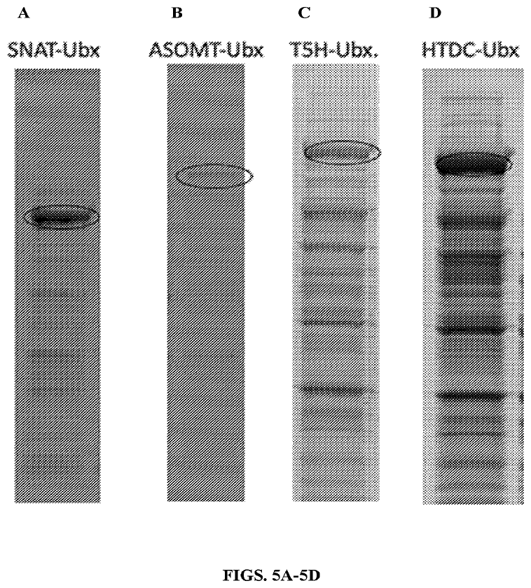

The second pathway immobilized four (4) enzymes (Serotonin N-AcetylTransferase-Ultrabithorax (SNAT-Ubx, 63 kDa), Acetylserotonin O-methyltransferase-Ultrabithorax (ASOMT-Ubx, 78 kDa), Tryptophan 5 Hydroxylase-Ultrabithorax (T5H-Ubx, 106 kDa), and 5-HydroxyTryptophan decarboxylase-Ultrabithorax (HTDC-Ubx, 104 kDa).) to Ubx materials and used three cofactors to produce melatonin from tryptophan (FIGS. 4 and 5A-5D). The cofactors and buffers used in this exemplary biocatalytic cascade are set forth in Table 3.

TABLE 3
Cofactors for production of Melatonin

| Enzyme | Substrate | Co-Factor | Product | Buffer |
|---|---|---|---|---|
| Tryptophan | L-tryptophan | 300 μM | 5- | 200 mM |
| hydroxylase | | tetrahydropterin | hydroxytryptophan | ammonium |
| | | | | sulfate |
| | | | | 7 mM DTT |
| | | | | 25 ug/ml |
| | | | | catalase |
| | | | | 25 PM |
| | | | | NH4Fe(II)SO4 |
| | | | | 50 mM |
| | | | | HEPES |
| | | | | 25° C. |
| 5- | 5- | 100 μM | Serotonin | 50 mM |
| hydroxytryptophan | hydroxytryptophan | pyridoxal | | phosphate, pH |
| decarboxylase | | phosphate | | 7.0 |
| | | | | 25° C. |
| Serotonin N | Serotonin | 1 mM acetyl- | N-acetylserotonin | 50 mM |
| Acyltransferase | | CoA | | phosphate, pH |
| | | | | 8.0 |
| | | | | 1 mM DTNB |
| | | | | 25° C. |
| Acylserotonin O- | N-acetylserotonin | 0.5 mM S- | Melatonin | 100 mM |
| methyltransferase | | Adenosyl-L- | | Phosphate, pH |
| | | Methionine | | 8.0 |
| | | | | 25° C. |

Both pathways used continuous flow production, as described above. The enzymes used in these pathways demonstrated that exemplary enzymes from the oxidoreductase, transferase, hydrolase, and lyase class of enzymes can all be immobilized on Ubx materials, and used to synthesize compounds in the systems and methods described herein.

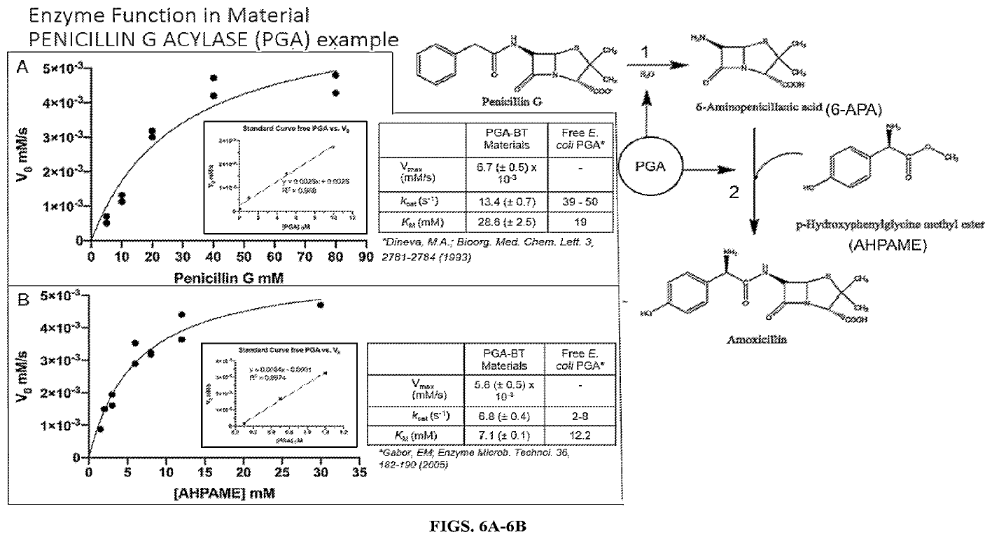

FIGS. 6A and 6B demonstrate that an enzyme or catalytic fragment fused to Ubx successfully catalyzes the desired reaction. Specifically, in this example, Penicillin G Acylase (PGA) catalyzes both steps in the conversion of Penicillin G to Amoxicillin. First, PGA catalyzes Penicillin G to 6-Aminopenicillanic acid (6-APA) (FIG. 6A), and subsequently, the condensation to Amoxicillin, through addition of p-hydroxyphenylglycine methyl ester (AHPAME) to the reaction (FIG. 6B).

Penicillin G has absorbance at 265 nm therefore, Penicillin G hydrolysis to 6-APA was measured by the disappearance of Penicillin G at 265 nm. The reaction occurred in 50 mM phosphate, pH 7.0, at 30° C., with enzyme-Ubx material wrapped around an innoculation loop, in a 1 mL quartz cuvette. The reaction was run at of 0.1, 1, 5, and 10 μM substrate concentrations and the reaction monitored. Absorbance was recorded for 900 seconds. Absorbance was converted to concentration using Beer's law, with the extinction coefficient equal to 119.8 M⁻¹ cm⁻¹. The data was plotted, normalized to the amount of material (enzyme) and linear regression was used to calculate enzymatic rate for each substrate concentration. The Vₘₐₓ and Kₘ were calculated by plotting the initial velocity for each concentration with the non-linear Michaelis-Menton equation.

The second PGA reaction to synthesize amoxicillin was conducted in 50 mM phosphate, pH 7.0 4 mM 6-APA, and 2, 3, 6, 8, or 12 mM of co-factor AHPAME. To measure activity of PGA synthesis of Amoxicillin from 6-APA the reaction product was reacted (200 mM acetate buffer, pH 2.0, 0.10% pDAB and 10% of the reaction at each specified time point) with dimethylaminobenzylaldehyde (p-DAB) and the absorbance recorded at 415 nm. Absorbance was converted to concentration using Beer's law and the extinction coefficient equal to 991.2 M⁻¹ cm⁻¹. Calculation of Vₘₐₓ and Kₘ were calculated as described above. As shown in FIG. 5, both PGA reactions demonstrate reaction kinetics consistent with the literature and demonstrate the catalytic activity of enzymes in Ubx materials (Dineva, M. A.; Bioorg. Med. Chem. Lett. 3, 2781-2784 (1993); Gabor, E M; Enzyme Microb. Technol. 36, 182-190 (2005)).

To demonstrate activity of a ligase class enzyme, Acetyl-CoA Synthetase was immobilized onto Ubx materials via gene fusion. Acetyl-CoA synthetase ligates acetate and coenzyme A to form Acetyly-CoA, a component of the TCA cycle and fatty acid synthesis. In industry, Acetyl-CoA-derived chemicals are suitable for multiple applications in the food additive, medicine, agriculture, cosmetic, and chemical industries. The bio-production of acetyl-CoA-derived chemicals offers a green alternative to petroleum-based applications. The reaction occurred in a quartz cuvette with enzyme-Ubx material wrapped around an inoculation loop, as previously described. The reaction occurred with 30 μl of 5, 10, 15, or 20 mM coenzyme A, 50 μl 0.1 M ATP, 12.5 μl of 0.2 M MgCl₂, 30 μl of 0.2 M sodium acetate, and 50 μl of hydroxylamine solution. 450 μl of ferric-chloride was added to stop each reaction prior to absorbance measurement at 540 nm. Enzyme kinetics were obtained as previously described.

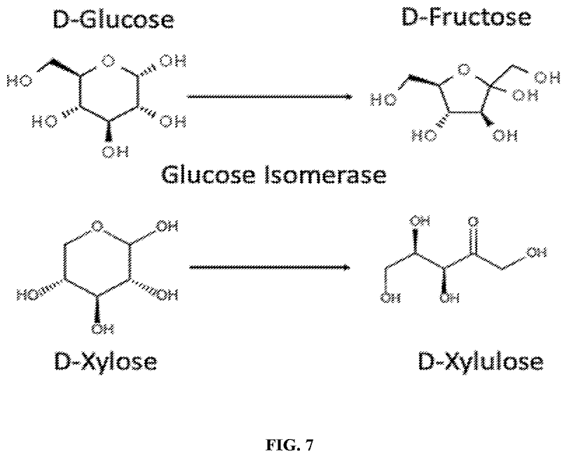

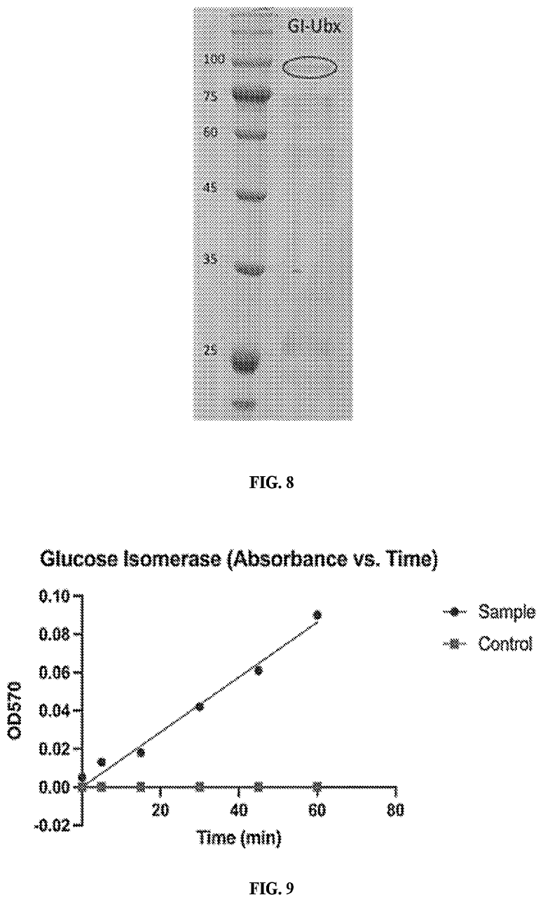

To demonstrate activity of isomerase in the systems and methods described, a test of the functionality of glucose isomerase fused to Ubx materials was conducted. This example demonstrated the ability to produce the valuable chemical fructose, for use in the food industry to produce high fructose corn syrup, from glucose via the enzyme glucose isomerase. Glucose isomerase (GI, an Isomerase class enzyme) is one of the most important industrial enzymes. However, GI requires low pH and high temperature for optimal activity, which can be overcome by immobilization to Ubx materials. In this example, GI was immobilized to Ubx materials to produce glucose from fructose (FIGS. 7 and 8). GI-Ubx materials were wrapped around an inoculation loop and tested for GI activity using the protocol and kit from Biovision (Glucose Isomerase Activity Assay kit, K491, Milpitas, CA). GI has a wide range of substrates, besides glucose, as it can catalyze the isomerization of xylose and other sugars. GI also converts xylose to xylulose, which can ultimately be fermented to ethanol by conventional yeasts (FIG. 7). Bioconversion of renewable biomass to fermentable sugars and ethanol is important as an alternate energy source. GI immobilized to Ubx materials can be economically suitable for use in the industrial production of valuable chemicals, such as fructose (FIG. 8) and ethanol.

### Enzyme Stability

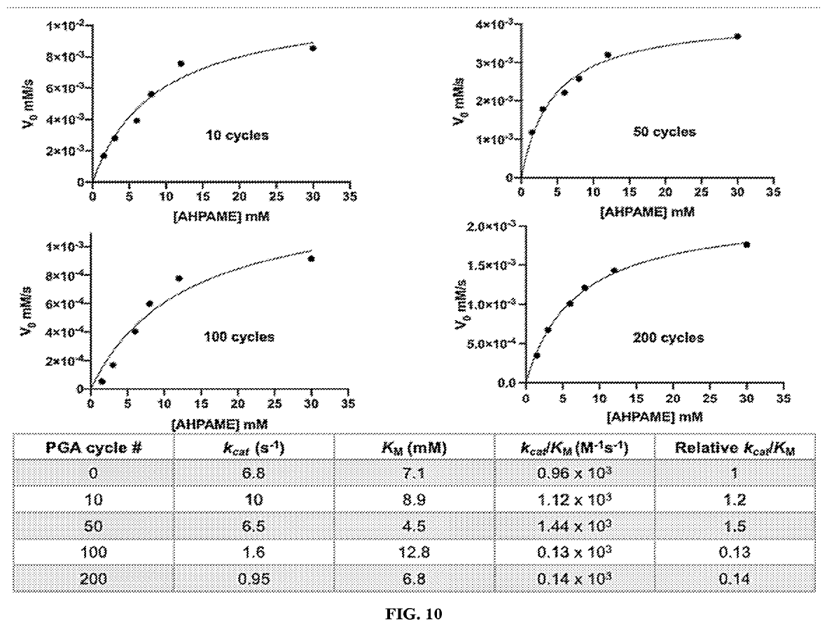

The immobilization of enzymes on solid supports can enhance enzyme stability to resist moderate increases in temperature or exposure to pH extremes or organic solvents. However, the increase in stability due to immobilization reported in the literature is insignificant compared to the stability conferred using the compositions and methods described herein. For example, dextran polyaldehyde or chitosan immobilized Penicillin G acylase has been shown to have an 18-fold increase in half-life at pH 10 (Mateo et al., Biotechnol. Bioeng. 86(3): 273-276 (2004), a 4.9-fold (Adriano et al., Braz. J Chem. Eng 22: 529-538 (2005) increase in stability at 50° C. for 4 hours, and a 2-fold increase when exposed to 60% dimethylformamide (Abian et al., Appl. Environ. Microbiol. 70(2): 1249-1251 (2004) compared to free enzyme. The immobilization technique described herein provides stability that has not been previously reported. Penicillin G Acylase-Ubx fibers were prepared as previously described, vacuum sealed in a 15 mL falcon tube and then exposed to conditions to mimic shipping and/or storage at temperatures fluctuating between 10° C.-30° C. Temperatures were ramped between 10° C.-30° C. over the course of 10, 50, 100, and 200 cycles of 2° C./minute. This Penicillin G Acylase-Ubx material activity was measured as previously described and showed that it retained its enzymatic activity as compared to fresh material, with reduced but similar k_{cat}, K_{M} and Vₘₐₓ values (FIG. 10).

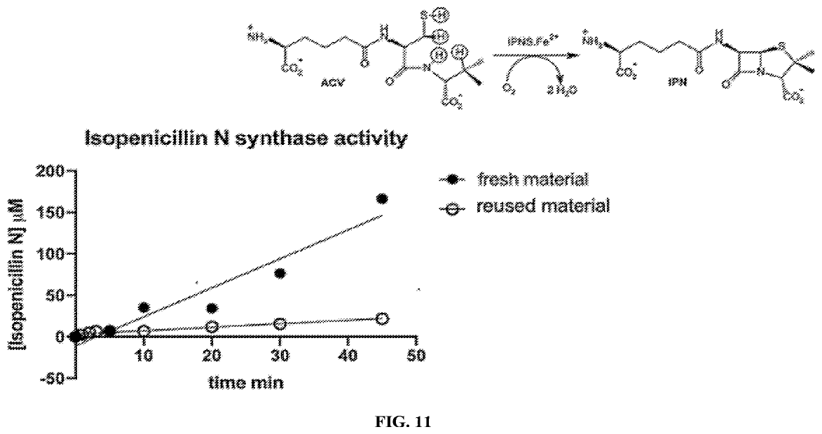

In another example, Isopenicillin N Synthase, fused with Ubx, was wrapped around an inoculation loop. The reaction conditions were 2 mM ACV, 1.2 mM TCEP, 50 mM Phosphate pH 7.0, 25 μM ascorbate, 10 μM NH₄Fe(II)SO₄ at 25° C. Isopenicillin N absorbs at 235 nm with the extinction coefficient of 1150 M⁻¹ cm⁻¹. Fresh material was analyzed for activity while additional material was stored dry and at room temperature for two days. Upon reuse, the material retained activity (FIG. 11). This demonstrates that the material can be reused at least twice. If the enzyme is stored in PBS instead of dried, the material retained significantly more activity.

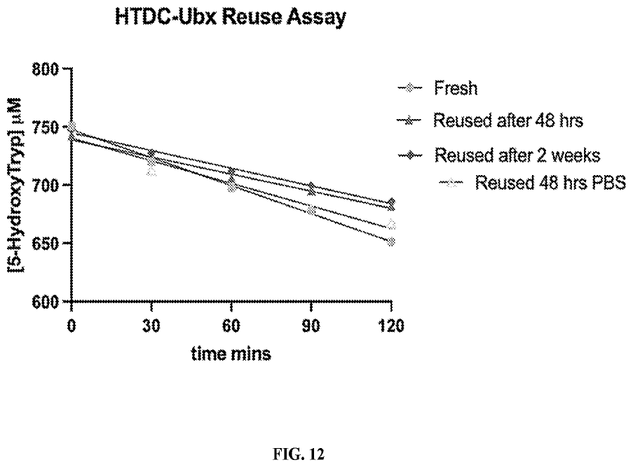

In this experiment, HTDC-Ubx materials were used to carry out the reaction of 5-HydroxyTryptophan to Serotonin (reaction conditions were 750 μM 5-HydroxyTryptophan, 50 mM phosphate pH 7.0, 7 mM DTT, 100 μM PLP at 25° C.; activity monitored by the disappearance of 5-HydroxyTryptophan at 330 nm on a fluorometer) and then, stored dry or in PBS at room temperature, for 48 hours. The reaction was run again using fresh substrate and the activity compared to the first run. Material stored in PBS retained 84% activity while material stored dry only retained 61% activity (FIG. 12). Therefore, storage in an aqueous buffer, like PBS, can contribute to material stability. Interestingly, there is no statistical difference between dry storage for 48 hours vs dry storage for 2 weeks.

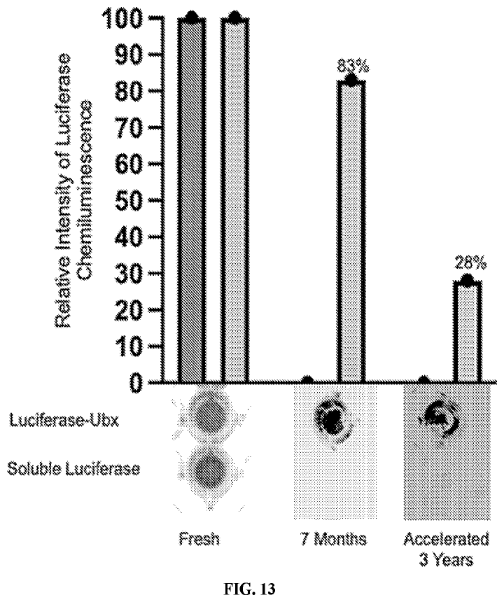

Another benefit of enzyme immobilization in Ubx materials is an increase in enzyme shelf-life. Soluble enzymes typically must be stored at sub-zero temperatures to remain active, but usually only have a shelf-life of several months. Therefore, there is a significant cost associated with storing enzymes until they are ready for use. The method of immobilization described in this invention allows enzymes to be stored at ambient temperature and retain activity (83%) even after 7 months (FIG. 13). In fact, using accelerated aging showed activity is retained in Luciferase-Ubx materials after 3 years (28% of fresh). Three years was simulated using an accelerated aging technique of placing the luciferase-Ubx material in an environmental chamber set to 40° C. and 55% humidity for 11 weeks. For comparison, soluble luciferase was used as a control and showed no signal after aging. The luciferase reaction was performed using the Promega Luciferase kit and chemiluminescent technology (Bio-rad ChemiDoc system, Hercules, CA).

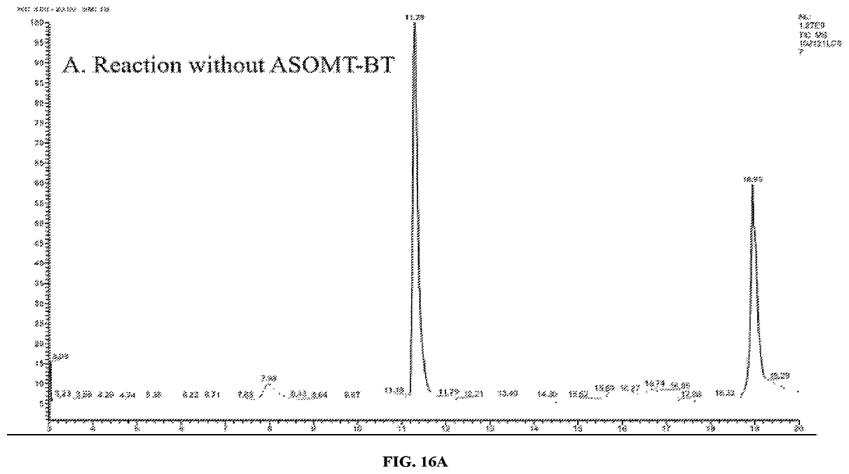

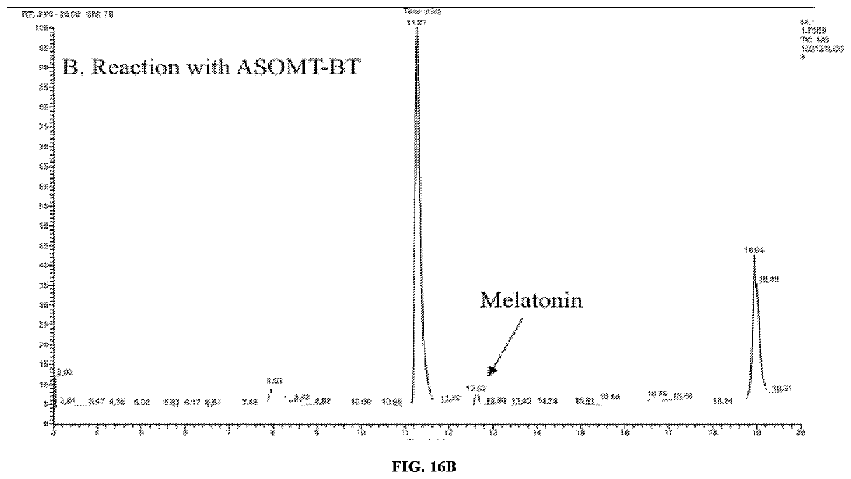

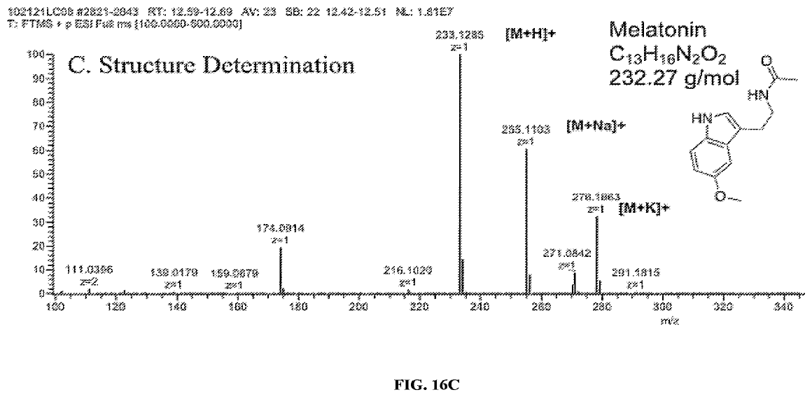

A method is demonstrated in which matrices, each modified with one or more enzymes are each sealed in a plastic reaction chamber. Therapeutics can be synthesized by connecting the appropriate series of reaction chambers, and adding substrate and any needed cofactors to the first reaction chamber. The desired final product will flow from the last chamber (FIG. 14). Each disk or matrix can be rinsed, dried, and re-used as needed to manufacture one or more drugs. This telescoping biocatalytic cascade method is demonstrated herein using the melatonin pathway. The reaction starts with tryptophan and uses 4 enzymes to catalyze melatonin (FIG. 4). The first three steps were completed in a single-pot reaction where Tryptophan 5 Hydroxylase-Ultrabithorax, 5-HydroxyTryptophan decarboxylase-Ultrabithorax, and Serotonin Acetylase (SNAT)-ultrabithorax materials are used together to catalyze N-acetylserotonin from tryptophan (FIG. 15). The reaction is monitored by detecting the presence of the by-product of SNAT generating N-acetylserotonin. The reaction conditions require 50 mM phosphate pH 7.5, 100 μM PLP, 300 μM tetrahydropterin, 200 mM (NH₄)₂SO₄, 25 μM NH₄Fe(II)SO₄, 25 μg/mL catalase, 1 mM DTNB, 1 mM Acetyl-CoA and 1 mM tryptophan at 25° C. The assay is monitored for N-acetylserotonin at 412 nm on a UV/Vis spectrophotometer. All three enzymes were required for reaction to occur, and no reaction was detected on single enzymes or a negative control with only Ubx materials and no enzyme present. 0.5 mM S-Adenosyl-L-Methionine was added to the product of this reaction (N-acetylserotonin) and used as the substrate for the final step of the pathway in a 2^{nd} reaction using Acetylserotonin O-methyltransferase-Ultrabithorax. The production of melatonin by this enzymatic cascade was confirmed with liquid chromatography-mass spectrometry (FIGS. 16A-C).

In summary, the systems, compositions, and methods described herein can be used to stabilize enzymes for use in one or more catalytic reactions to produce one or more desired compounds, including, but not limited to, a pharmaceutical, a biofuel, a fine chemical, or a bulk chemical.

### Exemplary Ubx and Enzyme-Ubx Fusion Polypeptide Sequences

Ubx
(SEQ ID NO: 5)
MNSYFEQASGFYGHPHQATGMAMGSGGHHDQTASAAAAAYRGFPLSLG
MSPYANHHLQRTTQDSPYDASITAACNKIYGDGAGAYKQDCLNIKADAV
NGYKDIWNTGGSNGGGGGGGGGGGGGAGGTGGAGNANGGNAANANGQNN
PAGGMPVRPSACTPDSRVGGYLDTSGGSPVSHRGGSAGGNVSVSGGNGN
AGGVQSGVGVAGAGTAWNANCTISGAAAQTAAASSLHQASNHTFYPWMA
IAGKIRSDLTQYGGISTDMGKRYSESLAGSLLPDWLGTNGLRRRGRQTY
TRYQTLELEKEFHTNHYLTRRRRIEMAHALCLTERQIKIWFQNRRMKLK
KEIQAIKELNEQEKQAQAQKAAAAAAAAAAVQGGHLDQ

In each of the amino acid sequences for the enzyme-Ubx fusion proteins set forth below (SEQ ID NOs: 15-28), the amino acid sequence of the enzyme (unmodified, unitalicized font), is followed by a linker (underlined), and the amino acid sequence of the Ubx polypeptide (italicized)

Penicillin G Acylase (PGA)-Ubx
(SEQ ID NO: 15)
MKNRNRMIVNCVTASLMYYWSLPALAEQSSSEIKIVRDEYGMPHIYAND
TWHLFYGYGYVVAQDRLFQMEMARRSTQGTVAEVLGKDFVKFDKDIRRNYWPDAI
RAQIAALSPEDMSILQGYADGMNAWIDKVNTNPETLLPKQFNTFGFTPKRWEPFDV
AMIFVGTMANRFSDSTSEIDNLALLTALKDKYGVSQGMAVFNQLKWLVNPSAPTTI
AVQESNYPLKFNQQNSQTAALLPRYDLPAPMLDRPAKGADGALLALTAGKNRETIA
AQFAQGGANGLAGYPTTSNMWVIGKSKAQDAKAIMVNGPQFGWYAPAYTYGIGL
HGAGYDVTGNTPFAYPGLVFGHNGVISWGSTAGFGDDVDIFAERLSAEKPGYYLHN
GKWVKMLSREETITVKNGQAETFTVWRTVHGNILQTDQTTQTAYAKSRAWDGKEV
ASLLAWTHQMKAKNWQEWTQQAAKQALTINWYYADVNGNIGYVHTGAYPDRQS
GHDPRLPVPGTGKWDWKGLLPFEMNPKVYNPQSGYIANWNNSPQKDYPASDLFAF
LWGGADRVTEIDRLLEQKPRLTADQAWDVIRQTSRQDLNLRLFLPTLQAATSGLTQS
DPRRQLVETLTRWDGINLLNDDGKTWQQPGSAILNVWLTSMLKRTVVAAVPMPFD
KWYSASGYETTQDGPTGSLNISVGAKILYEAVQGDKSPIPQAVDLFAGKPQQEVVLA
ALEDTWETLSKRYGNNVSNWKTPAMALTFRANNFFGVPQAAAEETRHQAEYQNRG
TENDMIVFSPTTSDRPVLAWDVVAPGQSGFIAPDGTVDKHYEDQLKMYENFGRKSL
WLTKQDVEAHKESQEVLHVQRTSGSGSHMNSYFEQASGFYGHPHQATGMAMGSGGH
HDQTASAAAAAYRGFPLSLGMSPYANHHLQRTTQDSPYDASITAACNKIYGDGAGAYKQD
CLNIKADAVNGYKDIWNTGGSNGGGGGGGGGGGGGAGGTGGAGNANGGNAANANGQ
NNPAGGMPVRPSACTPDSRVGGYLDTSGGSPVSHRGGSAGGNVSVSGGNGNAGGVQSG
VGVAGAGTAWNANCTISGAAAQTAAASSLHQASNHTFYPWMAIAGKIRSDLTQYGGISTD
MGKRYSESLAGSLLPDWLGTNGLRRRGRQTYTRYQTLELEKEFHTNHYLTRRRRIEMAHA
LCLTERQIKIWFQNRRMKLKKEIQAIKELNEQEKQAQAQKAAAAAAAAAAVQGGHLDQ
Isopenicillin N synthase (IPNS)-Ubx
(SEQ ID NO: 16)
MASTPKANVPKIDVSPLFGDNMEEKMKVARAIDAASRDTGFFYAVNHGV
DVKRLSNKTREFHFSITDEEKWDLAIRAYNKEHQDQIRAGYYLSIPEKKAVESFCYL
NPNFKPDHPLIQSKTPTHEVNVWPDEKKHPGFREFAEQYYWDVFGLSSALLRGYAL
ALGKEEDFFSRHFKKEDALSSVVLIRYPYLNPIPPAAIKTAEDGTKLSFEWHEDVSLIT
VLYQSDVANLQVEMPQGYLDIEADDNAYLVNCGSYMAHITNNYYPAPIHRVKWVN
EERQSLPFFVNLGFNDTVQPWDPSKEDGKTDQRPISYGDYLQNGLVSLINKNGQTTS
GSGSHMNSYFEQASGFYGHPHQATGMAMGSGGHHDQTASAAAAAYRGFPLSLGMSPYA
NHHLQRTTQDSPYDASITAACNKIYGDGAGAYKQDCLNIKADAVNGYKDIWNTGGSNGG
GGGGGGGGGGGAGGTGGAGNANGGNAANANGQNNPAGGMPVRPSACTPDSRVGGYL
DTSGGSPVSHRGGSAGGNVSVSGGNGNAGGVQSGVGVAGAGTAWNANCTISGAAAQTAA
ASSLHQASNHTFYPWMALAGKIRSDLTQYGGISTDMGKRYSESLAGSLLPDWLGTNGLRRR
GRQTYTRYQTLELEKEFHTNHYLTRRRRIEMAHALCLTERQIKIWFQNRRMKLKKEIQAIK
ELNEQEKQAQAQKAAAAAAAAAAVQGGHLDQ
Isopenicillin N Epimerase (IPNE)-Ubx
(SEQ ID NO: 17)
MAVADWEEARGRMLLDPTVVNLNTGSGGPLPRSAFERVTGFRAHLAAEP
MDFLLREVPALLWQARESLARLIGGDPLRLALATNVTAAVNLVASSLRLEAPGEILL
SDDEYTPMRWCWERVARRHGLELRTFRLPELPSDPAEITAAAVAAMGPRTRLFFFSH
VVSTTGLILPAAELCEEARARGITTVVDGAHAPGFLDLDLSRIPCDFYAGSGHKWLL
APTGVGFLHLAPGRLEELEPTQVSWAYEPPEGSGPPAARDRFGSTPGLRRLECEGTR
DICPWLATPESIDFQAELGPGAIRARRRELTDHARRLLADRPGRTLLTPDSPELSGGM
VAYRLPPGTDAAELRRGLWERFRIEAAVAEQPPGPVLRISANFYTTEEEIDRLADALD
ALTGETSGSGSHMNSYFEQASGFYGHPHQATGMAMGSGGHHDQTASAAAAAYRGFPLS
LGMSPYANHHLQRTTQDSPYDASITAACNKIYGDGAGAYKQDCLNIKADAVNGYKDIWNT
GGSNGGGGGGGGGGGGGAGGTGGAGNANGGNAANANGQNNPAGGMPVRPSACTPDS
RVGGYLDTSGGSPVSHRGGSAGGNVSVSGGNGNAGGVQSGVGVAGAGTAWNANCTISGA
AAQTAAASSLHQASNHTFYPWMAIAGKIRSDLTQYGGISTDMGKRYSESLAGSLLPDWLGT
NGLRRRGRQTYTRYQTLELEKEFHTNHYLTRRRRIEMAHALCLTERQIKIWFQNRRMKLK
KEIQAIKELNEQEKQAQAQKAAAAAAAAAAVQGGHLDQ
Deacetoxycephalosporin C synthetase (DAOCS)-Ubx
(SEQ ID NO: 18)
MDTTVPTFSLAELQQGLHQDEFRRCLRDKGLFYLTDCGLTDTELKSAKDL
VIDFFEHGSEAEKRAVTSPVPTMRRGFTGLESESTAQITNTGSYSDYSMCYSMGTAD
NLFPSGDFERIWTQYFDRQYTASRAVAREVLRATGTEPDGGVEAFLDCEPLLRFRYF
PQVPEHRSAEEQPLRMAPHYDLSMVTLIQQTPCANGFVSLQAEVGGAFTDLPYRPDA
VLVFCGAIATLVTGGQVKAPRHHVAAPRRDQIAGSSRTSSVFFLRPNADFTFSVPLAR
ECGFDVSLDGETATFQDWIGGNYVNIRRTSKATSGSGSHMNSYFEQASGFYGHPHQA
TGMAMGSGGHHDQTASAAAAAYRGFPLSLGMSPYANHHLQRTTQDSPYDASITAACNKIY
GDGAGAYKQDCLNIKADAVNGYKDIWNTGGSNGGGGGGGGGGGGGAGGTGGAGNAN
GGNAANANGQNNPAGGMPVRPSACTPDSRVGGYLDTSGGSPVSHRGGSAGGNVSVSGG
NGNAGGVQSGVGVAGAGTAWNANCTISGAAAQTAAASSLHQASNHTFYPWMAIAGKIRSD
LTQYGGISTDMGKRYSESLAGSLLPDWLGTNGLRRRGRQTYTRYQTLELEKEFHTNHYLTR
RRRIEMAHALCLTERQIKIWFQNRRMKLKKEIQAIKELNEQEKQAQAQKAAAAAAAAAAV
QGGHLDQ
Deacetylcephalosporin C synthetase (DACS)-Ubx
(SEQ ID NO: 19)
MADTPVPIFNLAALREGADQEKFRECVTGMGVFYLTGYGAGDKDHRLAT
DTAMDFFANGTEAEKAAVTTDVPTMRRGYSALEAESTAQVTRTGSYTDYSMSFSM
GISGNVFPSPEFERVWTEYFDKLYAAAQETARLVLTASGGYDAEIVGSLDELLDADP
VLRLRYFPEVPEHRSAEHEPRRMAPHYDLSIITFIHQTPCANGFVSLQAEIGGELVSLP
VVEDAVVVMCGAMAPLATQGALPAPRHHVRSPGAGMREGSDRTSSVFFLRPTTDFS
FSVAKARSYGLAVDLDMETATFGDWIGTNYVTMHAKNEPQAGTSGSGSHMNSYFE
QASGFYGHPHQATGMAMGSGGHHDQTASAAAAAYRGFPLSLGMSPYANHHLQRTTQDS
PYDASITAACNKIYGDGAGAYKQDCLNIKADAVNGYKDIWNTGGSNGGGGGGGGGGGG
GAGGTGGAGNANGGNAANANGQNNPAGGMPVRPSACTPDSRVGGYLDTSGGSPVSHRG
GSAGGNVSVSGGNGNAGGVQSGVGVAGAGTAWNANCTISGAAAQTAAASSLHQASNHTF
YPWMAIAGKIRSDLTQYGGISTDMGKRYSESLAGSLLPDWLGTNGLRRRGRQTYTRYQTLE
LEKEFHTNHYLTRRRRIEMAHALCLTERQIKIWFQNRRMKLKKEIQAIKELNEQEKQAQA
QKAAAAAAAAAAVQGGHLDQ
Deacetylcephalosporin C acetyltransferase DACOAT-Ubx
(SEQ ID NO: 20)
MLPSAQVARLKPDPFPPSLSPIPHGAVTFAALAPCHNLPIFSSRQMLRDSLT
YSHTSPTMSPQIANRFEASLDAQDIARISLFTLESGVILRDVPVAYKSWGRMNVSRDN
CVIVCHTLTSSAHVTSWWPTLFGQGRAFDTSRYFIICLNYLGSPFGSAGPCSPDPDAE
GQRPYGAKFPRTTIRDDVRIHRQVLDRLGVRQIAAVVGASMGGMHTLEWAFFGPEY
VRKIVPIATSCRQSGWCAAWFETQRQCIYDDPKYLDGEYDVDDQPVRGLETARKIA
NLTYKSKPAMDERFHMAPGVQAGRNISSQDAKKEINGTDSGNSHRAGQPIEAVSSYL
RYQAQKFAASFDANCYIAMTLKFDTHDISRGRAGSIPEALAMITQPALIICARSDGLY
SFDEHVEMGRSIPNSRLCVVDTNEGHDFFVMEADKVNDAVRGFLDQSLMTSGSGSH
MNSYFEQASGFYGHPHQATGMAMGSGGHHDQTASAAAAAYRGFPLSLGMSPYANHHLQ
RTTQDSPYDASITAACNKIYGDGAGAYKQDCLNIKADAVNGYKDIWNTGGSNGGGGGGG
GGGGGGAGGTGGAGNANGGNAANANGQNNPAGGMPVRPSACTPDSRVGGYLDTSGGS
PVSHRGGSAGGNVSVSGGNGNAGGVQSGVGVAGAGTAWNANCTISGAAAQTAAASSLHQ
ASNHTFYPWMALAGKIRSDLTQYGGISTDMGKRYSESLAGSLLPDWLGTNGLRRRGRQTYT
RYQTLELEKEFHTNHYLTRRRRIEMAHALCLTERQIKIWFQNRRMKLKKEIQAIKELNEQE
KQAQAQKAAAAAAAAAAVQGGHLDQ
Isopenicillin N acyltranferase (IPNAT)-Ubx (IPNAT from
Streptomyces clavuligerus)
(SEQ ID NO: 21)
MATAPLVIRAEGGPRERGARYGAAARDKVRMSLGSYERVYAKFAGMSW
TEATTLAGEFVPVLSGWRPEYLEEIRGIAEGAGVSFEDVLALNLRTEIMFSGKARRLA
EETGIRPPAECTAFCDLRGPEPLAGQNWDWIPFAHDTVVVLETAPERGPRWVSVVEA
GLLAKFGMNSAGIAVVTNALVTSLDVGAPGVPYHMLLRALLESESMAAAEEVLRG
ADRASSANYLLVGEGRGVDAEARPGGVGDLGWRRTGAEAVLVHCNHFVCAEAEFS
SSGTDHGPEIMPDSLLRRERALSLAGRMPEPSVDGWHEVLTDHENFPAAVCCHPHPE
RTALEQGSTVASVVMEPERRLMHLVLGRPCEAPRVTYDYGTFLAGTSGSGSHMNSY
FEQASGFYGHPHQATGMAMGSGGHHDQTASAAAAAYRGFPLSLGMSPYANHHLQRTTQ
DSPYDASITAACNKIYGDGAGAYKQDCLNIKADAVNGYKDIWNTGGSNGGGGGGGGGG
GGGAGGTGGAGNANGGNAANANGQNNPAGGMPVRPSACTPDSRVGGYLDTSGGSPVS
HRGGSAGGNVSVSGGNGNAGGVQSGVGVAGAGTAWNANCTISGAAAQTAAASSLHQASN
HTFYPWMAIAGKIRSDLTQYGGISTDMGKRYSESLAGSLLPDWLGTNGLRRRGRQTYTRY
QTLELEKEFHTNHYLTRRRRIEMAHALCLTERQIKIWFQNRRMKLKKEIQAIKELNEQEKQ
AQAQKAAAAAAAAAAVQGGHLDQ
Tryptophan hydroxylase (TPH)-Ubx
(SEQ ID NO: 22)
MQPAMMMFSSKYWARRGFSLDSAVPEEHQLLGSSTLNKPNSGKNDDKG
NKGSSKREAATESGKTAVVFSLKNEVGGLVKALRLFQEKRVNMVHIESRKSRRRSSE
VEIFVDCECGKTEFNELIQLLKFQTTIVTLNPPENIWTEEEELEDVPWFPRKISELDKCS
HRVLMYGSELDADHPGFKDNVYRQRRKYFVDVAMGYKYGQPIPRVEYTEEETKTW
GVVFRELSKLYPTHACREYLKNFPLLTKYCGYREDNVPQLEDVSMFLKERSGFTVRP
VAGYLSPRDFLAGLAYRVFHCTQYIRHGSDPLYTPEPDTCHELLGHVPLLADPKFAQ
FSQEIGLASLGASDEDVQKLATCYFFTIEFGLCKQEGQLRAYGAGLLSSIGELKHALS
DKACVKAFDPKTTCLQECLITTFQEAYFVSESFEEAKEKMRDFAKSITRPFSVYFNPY
TQSIEILKDTRSIENVVQDLRSDLNTVCDALNKMNQYLGITSGSGSHMNSYFEQASGF
YGHPHQATGMAMGSGGHHDQTASAAAAAYRGFPLSLGMSPYANHHLQRTTQDSPYDASI
TAACNKIYGDGAGAYKQDCLNIKADAVNGYKDIWNTGGSNGGGGGGGGGGGGGAGGT
GGAGNANGGNAANANGQNNPAGGMPVRPSACTPDSRVGGYLDTSGGSPVSHRGGSAGG
NVSVSGGNGNAGGVQSGVGVAGAGTAWNANCTISGAAAQTAAASSLHQASNHTFYPWMA
LAGKIRSDLTQYGGISTDMGKRYSESLAGSLLPDWLGTNGLRRRGRQTYTRYQTLELEKEF
HTNHYLTRRRRIEMAHALCLTERQIKIWFQNRRMKLKKEIQAIKELNEQEKQAQAQKAAA
AAAAAAAVQGGHLDQ
5-HydroxyTryptophan decarboxylase (HTDC)-Ubx
(SEQ ID NO: 23)
MNASEFRRRGKEMVDYMANYMEGIEGRQVYPDVEPGYLRPLIPAAAPQE
PDTFEDIINDVEKIIMPGVTHWHSPYFFAYFPTASSYPAMLADMLCGAIGCIGFSWAA
SPACTELETVMMDWLGKMLELPKAFLNEKAGEGGGVIQGSASEATLVALLAARTK
VIHRLQAASPELTQAAIMEKLVAYSSDQAHSSVERAGLIGGVKLKAIPSDGNFAMRA
SALQEALERDKAAGLIPFFMVATLGTTTCCSFDNLLEVGPICNKEDIWLHVDAAYAG
SAFICPEFRHLLNGVEFADSFNFNPHKWLLVNFDCSAMWVKKRTDLTGAFRLDPTY
LKHSHQDSGLITDYRHWQIPLGRRFRSLKMWFVFRMYGVKGLQAYIRKHVQLSHEF
ESLVRQDPRFEICVEVILGLVCFRLKGSNKVNEALLQRINSAKKIHLVPCHLRDKFVL
RFAICSRTVESAHVQRAWEHIKELAADVLRAERETSGSGSHMNSYFEQASGFYGHPH
QATGMAMGSGGHHDQTASAAAAAYRGFPLSLGMSPYANHHLQRTTQDSPYDASITAACN
KIYGDGAGAYKQDCLNIKADAVNGYKDIWNTGGSNGGGGGGGGGGGGGAGGTGGAGN
ANGGNAANANGQNNPAGGMPVRPSACTPDSRVGGYLDTSGGSPVSHRGGSAGGNVSVS
GGNGNAGGVQSGVGVAGAGTAWNANCTISGAAAQTAAASSLHQASNHTFYPWMAIAGKI
RSDLTQYGGISTDMGKRYSESLAGSLLPDWLGTNGLRRRGRQTYTRYQTLELEKEFHTNH
YLTRRRRIEMAHALCLTERQIKIWFQNRRMKLKKEIQAIKELNEQEKQAQAQKAAAAAAAA
AAVQGGHLDQ
Serotonin Acetylase (SNAT)-Ubx
(SEQ ID NO: 24)
MSTQSTHPLKPEAPRLPPGIPESPSCQRRHTLPASEFRCLTPEDAVSAFEIER
EAFISVLGVCPLYLDEIRHFLTLCPELSLGWFEEGCLVAFIIGSLWDKERLMQESLTLH
RSGGHIAHLHVLAVHRAFRQQGRGPILLWRYLHHLGSQPAVRRAALMCEDALVPFY
ERFSFHAVGPCAITVGSLTFMELHCSLRGHPFLRRNSGCTSGSGSHMNSYFEQASGFY
GHPHQATGMAMGSGGHHDQTASAAAAAYRGFPLSLGMSPYANHHLQRTTQDSPYDASIT
AACNKIYGDGAGAYKQDCLNIKADAVNGYKDIWNTGGSNGGGGGGGGGGGGGAGGTG
GAGNANGGNAANANGQNNPAGGMPVRPSACTPDSRVGGYLDTSGGSPVSHRGGSAGGN
VSVSGGNGNAGGVQSGVGVAGAGTAWNANCTISGAAAQTAAASSLHQASNHTFYPWMAI
AGKIRSDLTQYGGISTDMGKRYSESLAGSLLPDWLGTNGLRRRGRQTYTRYQTLELEKEFH
TNHYLTRRRRIEMAHALCLTERQIKIWFQNRRMKLKKEIQAIKELNEQEKQAQAQKAAAA
AAAAAAVQGGHLDQ
Acetylserotonin O-methyltransferase (ASOMT)-Ubx
(SEQ ID NO: 25)
MGSSEDQAYRLLNDYANGFMVSQVLFAACELGVFDLLAEAPGPLDVAA
VAAGVRASAHGTELLLDICVSLKLLKVETRGGKAFYRNTELSSDYLTTVSPTSQCSM
LKYMGRTSYRCWGHLADAVREGRNQYLETFGVPAEELFTAIYRSEGERLQFMQALQ
EVWSVNGRSVLTAFDLSVFPLMCDLGGGAGALAKECMSLYPGCKITVFDIPEVVWT
AKQHFSFQEEEQIDFQEGDFFKDPLPEADLYILARVLHDWADGKCSHLLERIYHTCK
PGGGILVIESLLDEDRRGPLLTQLYSLNMLVQTEGQERTPTHYHMLLSSAGFRDFQFK
KTGAIYDAILARKTSGSGSHMNSYFEQASGFYGHPHQATGMAMGSGGHHDQTASAAA
AAYRGFPLSLGMSPYANHHLQRTTQDSPYDASITAACNKIYGDGAGAYKQDCLNIKADAV
NGYKDIWNTGGSNGGGGGGGGGGGGGAGGTGGAGNANGGNAANANGQNNPAGGMP
VRPSACTPDSRVGGYLDTSGGSPVSHRGGSAGGNVSVSGGNGNAGGVQSGVGVAGAGTA
WNANCTISGAAAQTAAASSLHQASNHTFYPWMAIAGKIRSDLTQYGGISTDMGKRYSESLA
GSLLPDWLGTNGLRRRGRQTYTRYQTLELEKEFHTNHYLTRRRRIEMAHALCLTERQIKIW
FQNRRMKLKKEIQAIKELNEQEKQAQAQKAAAAAAAAAAVQGGHLDQ
Glucose Isomerase-Ubx
(SEQ ID NO: 26)
MSKYFEKVSKIKYEGPKSNNPYAFKFYNPEEVIDGKTMEEHLRFSIAYWH
TFTADGTDQFGKATMQRPWNHLTDPMDIAKARVEAAFEFFDKINAPFFCFHDRDIAP
EGDTLRETNKNLDIIVAMIKDYLKTSKTKVLWGTANLFSNPRFVHGASTSCNADVFA
YSAAQVKKALEITKELGGQNYVFWGGREGYETLLNTDMELELDNFARFLHMAVDY
AKEIGFEGQFLIEPKPKEPTKHQYDFDVANVLAFLRKYDLDKYFKVNIEANHATLAA
HDFQHELRYARINGVLGSIDANTGDMLLGWDTDQFPTDIRMTTLAMYEVIKMGGFD
KGGLNFDAKVRRASFEPEDLFLGHIAGMDAFAKGFKVAYKLVKDGVFDKFIEERYA
SYKDGIGADIVSGKADFKSLEKYALEHSEIVNKSGRQEMLESILNQYLFTETSGSGSH
MNSYFEQASGFYGHPHQATGMAMGSGGHHDQTASAAAAAYRGFPLSLGMSPYANHHLQ
RTTQDSPYDASITAACNKIYGDGAGAYKQDCLNIKADAVNGYKDIWNTGGSNGGGGGGG
GGGGGGAGGTGGAGNANGGNAANANGQNNPAGGMPVRPSACTPDSRVGGYLDTSGGS
PVSHRGGSAGGNVSVSGGNGNAGGVQSGVGVAGAGTAWNANCTISGAAAQTAAASSLHQ
ASNHTFYPWMAIAGKIRSDLTQYGGISTDMGKRYSESLAGSLLPDWLGTNGLRRRGRQTYT
RYQTLELEKEFHTNHYLTRRRRIEMAHALCLTERQIKIWFQNRRMKLKKEIQAIKELNEQE
KQAQAQKAAAAAAAAAAVQGGHLDQ
Acetyl-CoA Synthetase
(SEQ ID NO: 27)
MSESTPEVSSSYPPPAHFAEHANARAELYREAEEDRLAFWAKQANRLSW
TTPFTEVLDWSGAPFAKWFVGGELNVAYNCVDRHVEAGHGDRVAIHWEGEPVGDR
RTLTYSDLLAEVSKAANALTDLGLVAGDRVAIYLPLIPEAVIAMLACARLGIMHSVV
FGGFTAAALQARIVDAQAKLLITADGQFRRGKPSPLKAAADEALAAIPDCSVEHVLV
VRRTGIEMAWSEGRDLWWHHVVGSASPAHTPEPFDSEHPLFLLYTSGTTGKPKGIM
HTSGGYLTQCCYTMRTIFDVKPDSDVFWCTADIGWVTGHTYGVYGPLCNGVTEVL
YEGTPDTPDRHRHFQIIEKYGVTIYYTAPTLIRMFMKWGREIPDSHDLSSLRLLGSVG
EPINPEAWRWYRDVIGGGRTPLVDTWWQTETGSAMISPLPGIAAAKPGSAMTPLPGI
SAKIVDDHGDPLPPHTEGAQHVTGYLVLDQPWPSMLRGIWGDPARYWHSYWSKFS
DKGYYFAGDGARIDPDGAIWVLGRIDDVMNVSGHRISTAEVESALVAHSGVAEAAV
VGVTDETTTQAICAFVVLRANYAPHDRTAEELRTEVARVISPIARPRDVHVVPELPKT
RSGKIMRRLLRDVAENRELGDTSTLLDPTVFDAIRAAKTSGSGSHMNSYFEQASGFYG
HPHQATGMAMGSGGHHDQTASAAAAAYRGFPLSLGMSPYANHHLQRTTQDSPYDASITA
ACNKIYGDGAGAYKQDCLNIKADAVNGYKDIWNTGGSNGGGGGGGGGGGGGAGGTGG
AGNANGGNAANANGQNNPAGGMPVRPSACTPDSRVGGYLDTSGGSPVSHRGGSAGGNV
SVSGGNGNAGGVQSGVGVAGAGTAWNANCTISGAAAQTAAASSLHQASNHTFYPWMALA
GKIRSDLTQYGGISTDMGKRYSESLAGSLLPDWLGTNGLRRRGRQTYTRYQTLELEKEFHT
NHYLTRRRRIEMAHALCLTERQIKIWFQNRRMKLKKEIQAIKELNEQEKQAQAQKAAAAA
AAAAAVQGGHLDQ
Luciferase-Ubx
(SEQ ID NO: 28)
MEDAKNIKKGPAPFYPLEDGTAGEQLHKAMKRYALVPGTIAFTDAHIEV
NITYAEYFEMSVRLAEAMKRYGLNTNHRIVVCSENSLQFFMPVLGALFIGVAVAPAN
DIYNERELLNSMNISQPTVVFVSKKGLQKILNVQKKLPIIQKIIIMDSKTDYQGFQSMY
TFVTSHLPPGFNEYDFVPESFDRDKTIALIMNSSGSTGLPKGVALPHRTACVRFSHAR
DPIFGNQIIPDTAILSVVPFHHGFGMFTTLGYLICGFRVVLMYRFEEELFLRSLQDYKI
QSALLVPTLFSFFAKSTLIDKYDLSNLHEIASGGAPLSKEVGEAVAKRFHLPGIRQGY
GLTETTSAILITPEGDDKPGAVGKVVPFFEAKVVDLDTGKTLGVNQRGELCVRGPMI
MSGYVNNPEATNALIDKDGWLHSGDLAYWDEDEHFFIVGRLKSLIKYKGYQVAPAE
LESILLQHPNIFDAGVAGLPDDDAGELPAAVVVLEHGKTMTEKEIVDYVASQVTTAK
KLRGGVVFVDEVPKGLTGKRDARKIREILIKAKKGGKSKLGHMNSYFEQASGFYGHP
HQATGMAMGSGGHHDQTASAAAAAYRGFPLSLGMSPYANHHLQRTTQDSPYDASITAAC
NKIYGDGAGAYKQDCLNIKADAVNGYKDIWNTGGSNGGGGGGGGGGGGGAGGTGGAG
NANGGNAANANGQNNPAGGMPVRPSACTPDSRVGGYLDTSGGSPVSHRGGSAGGNVSVS
GGNGNAGGVQSGVGVAGAGTAWNANCTISGAAAQTAAASSLHQASNHTFYPWMAIAGKI
RSDLTQYGGISTDMGKRYSESLAGSLLPDWLGTNGLRRRGRQTYTRYQTLELEKEFHTNH
YLTRRRRIEMAHALCLTERQIKIWFQNRRMKLKKEIQAIKELNEQEKQAQAQKAAAAAAAA
AAVQGGHLDQ

1. A system for catalytically producing a compound comprising: one or more solid matrices comprising a fusion protein, wherein the fusion proteins comprises at least one Ubx protein and an enzyme or catalytic fragment thereof.

2. The system of claim 1, wherein the system comprises, two, three, four, five, six, seven, or more solid matrices.

3. The system of claim 1, wherein the system comprises one or more reaction chambers comprising the one or more solid matrices.

4. The system of claim 3, wherein the system comprises two, three, four, five, six, or seven or more reaction chambers.

5. The system of claim 4, wherein the two, three, four, five, six, or seven or more reaction chambers are in fluid communication with each other.

6. The system of claim 2, wherein the enzymes or catalytic fragment thereof in the two, three, four, five, six, or seven or more solid matrices are enzymes in a biocatalytic cascade.

7. The system of claim 1, wherein the enzyme or catalytic fragment thereof is an oxidoreductase, a transferase, a hydrolase, a lyase, a ligase, an isomerase, or a catalytic fragment thereof.

8. The system of claim 1, wherein the enzyme or catalytic fragment thereof in each fusion protein is the same or different.

9. The system of claim 1, wherein the fusion protein in each chamber is immobilized.

10. The system of claim 1, wherein the enzyme or catalytic fragment of the fusion protein in each chamber is stabilized.

11. The system of claim 1, wherein the Ubx protein comprises an amino acid sequence having at least 90% identity to SEQ ID NO: 5, or a fragment thereof.

12. A method for producing a desired compound comprising:
(a) contacting a first reaction chamber of the one or more reaction chambers of the system of any of claim 3, with a substrate, to catalytically produce one or more reaction products; and
(b) (i) recovering the desired compound from the one or more reaction products produced in the first reaction chamber; or
(ii) continuously or non-continuously flowing the one or more reactions products from the first reaction chamber to one or more subsequent reaction chambers for further catalysis; and recovering the desired compound from the one or more subsequent reaction chambers.

13. The method of claim 12, wherein one or more cofactors are added to the first reaction chamber or to the one or more subsequent reaction chambers.

14. The method of claim 12, wherein the compound is a pharmaceutical, a biofuel, a fine chemical, or a bulk chemical.

15. The method of claim 14, wherein the pharmaceutical is an antibiotic or a hormone.

16. A fusion protein comprising at least one Ubx protein and an enzyme or catalytic fragment thereof wherein the enzyme or catalytic fragment thereof is an oxidoreductase, a transferase, a hydrolase, a lyase, a ligase, an isomerase, or a catalytic fragment thereof.

17. The fusion protein of claim 16, wherein the enzyme or catalytic fragment of the fusion protein has increased stability as compared to the enzyme or catalytic fragment when it is not fused to the Ubx protein.

18. A solid matrix comprising the fusion protein of claim 16.

19. A reaction chamber comprising the fusion protein of claim 16.

20. A reaction chamber comprising the solid matrix of claim 18.
